(19) United States
(12) Patent Application Publication
(10) Pub. No.: US 2020/0029872 A1
(43) Pub. Date: Jan. 30, 2020

(54) ARRAY OF TRANSPARENT BIOSENSORS INTEGRATED ON A TRANSPARENT SUBSTRATE, AND METHOD FOR FORMING SUCH

(71) Applicant: Oregon State University, Corvallis, OR (US)

(72) Inventors: Gregory S. Herman, Corvallis, OR (US); Xiasong Du, Corvallis, OR (US)

(73) Assignee: Oregon State University, Corvallis, OR (US)

(21) Appl. No.: 16/603,565

(22) PCT Filed: Apr. 20, 2018
(86) PCT No.: PCT/US18/28690

§ 371 Date: Oct. 7, 2019

Related U.S. Application Data
(60) Provisional application No. 62/488,670, filed on Apr. 21, 2017.

(51) Int. Cl. A61B 5/145; G01N 33/66; A61B 5/00; G01N 27/414

(52) U.S. Cl. CPC A61B 5/14532; A61B 5/14507; G01N 33/66; A61B 5/1486; G01N 27/4145; G01N 27/4146; A61B 5/6821

(57) ABSTRACT

An apparatus is provided which comprises: a substrate comprising a transparent material; and an array of transparent active devices disposed on the substrate, wherein each transparent active device comprises a sensing element associated with the transparent active device.

### CLAIM OF PRIORITY

This Application claims priority to U.S. Provisional Patent Application No. 62/448,670, filed 21 Apr. 2017, and titled, “ARRAY OF TRANSPARENT BIOSENSORS INTEGRATED ON A TRANSPARENT SUBSTRATE, AND METHOD FOR FORMING SUCH”, and which is incorporated by reference in its entirety for all purposes.

### BACKGROUND

A wide range of diagnostic potential exists through monitoring tears in the human eye. Continuous monitoring of chemicals, metabolites, proteins, antibodies, and other biomarkers in tears are of interest since tears are readily assessable and a less complex body fluid compared to serum or plasma. Previous attempts to integrate biosensors into contact lenses has been limited to only one or two sensors, that were externally powered and were an obstacle in seeing clearly due to their non-transparent nature. Further, the non-transparent nature of associated components of the biosensors such as antenna, controller circuitry, storage system, etc. limit functionality of integrating biosensors into contact lens by blocking vision.

### BRIEF DESCRIPTION OF THE DRAWINGS

The embodiments of the disclosure will be understood more fully from the detailed description given below and from the accompanying drawings of various embodiments of the disclosure, which, however, should not be taken to limit the disclosure to the specific embodiments, but are for explanation and understanding only.

FIGS. 1A-D illustrate a process of micro-contact printing on a curved substrate, according to some embodiments.

FIG. 1E illustrates an optical image of an Amorphous indium (In) gallium (Ga) zinc (Zn) oxide (O) (IGZO) field effect transistors (FET), according to some embodiments.

FIG. 1F illustrates a schematic of an IGZO FET structure on a curved substrate, according to some embodiments.

FIG. 1G illustrates a Scanning Electron Microscopy (SEM) image of a step edge between an Indium Tin Oxide (ITO) and substrate and between an IGZO/ITO and IGZO, according to some embodiments.

FIGS. 2A-B illustrate plots showing output characteristics and transfer characteristics of an IGZO-FET (W/L ratio of 100 um/20 um) on a curved substrate fabricated by μCP, respectively, according to some embodiments.

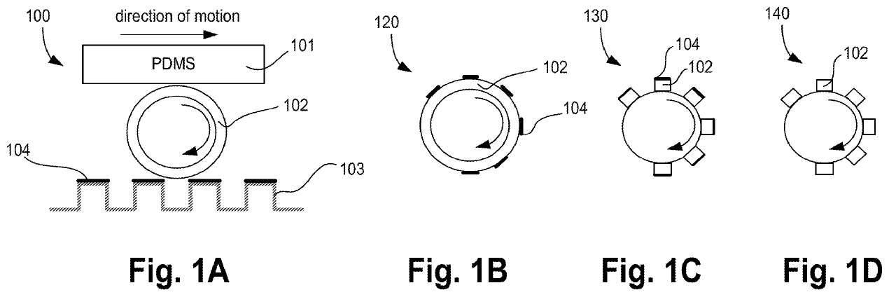

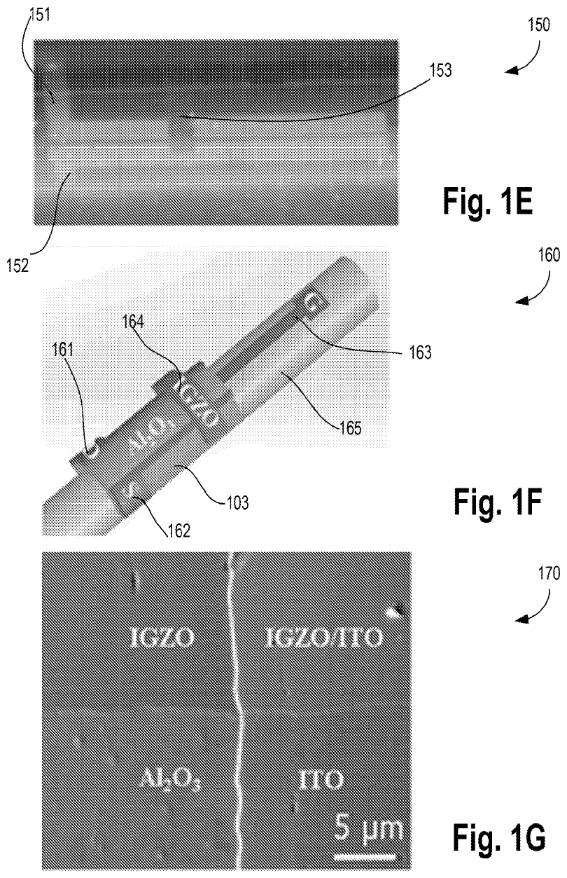

FIGS. 1A-B illustrate plots showing the difference in drain current (AID) versus time for various glucose concentrations as measured using functionalized IGZO-FET on curved substrate, and AID versus glucose concentration on a logarithmic scale, according to some embodiments.

FIG. 4 illustrates a three dimensional view of an array of IGZO FET based biosensors integrated in contact lens, according to some embodiments.

FIGS. 5A-C illustrate plots showing transfer characteristics of IGZO-FET test structures with different backchannel chemistries that are fabricated using a heavily p-doped Si substrate as the gate and thermally grown Sift (e.g., 100 nm thick) as the gate dielectric, according to some embodiments.

FIGS. 6-8 illustrate cross-sections of various embodiments of the IGZO-FET structure, according to some embodiments.

FIG. 9A illustrates a 3D illustration of an apparatus comprising an IGZO-FET sensor, in accordance with some embodiments.

FIG. 9B illustrates an optical image of IGZO-FET device with W/L ratio of 100 μm/20 μm, in accordance with some embodiments.

FIGS. 10A-B illustrate plots showing IGZO-FET backchannel functionalized with aminopropyltrimethoxysilane (ATPMS) exposed to varying pH, and source (S) and drain (D) current vs. pH, in accordance with some embodiments.

FIG. 11A illustrates a plot of an IGZO-FET backchannel functionalized with GOx exposed to varying concentrations of glucose, inset, and including a schematic diagram showing the role of positively charged aminosilane groups as an electron acceptor, according to some embodiments.

FIG. 11B illustrates a schematic diagram showing the impact of positively charged aminosilane groups on band bending at IGZO surface, according to some embodiments.

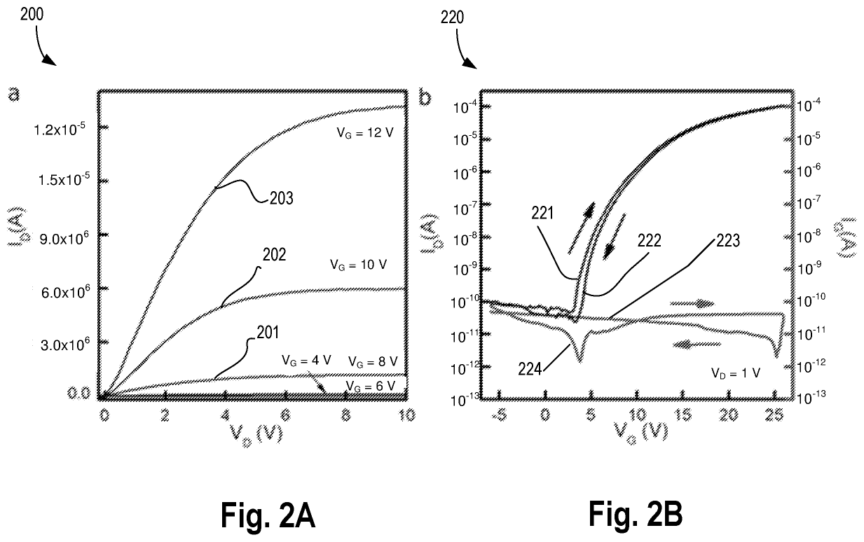

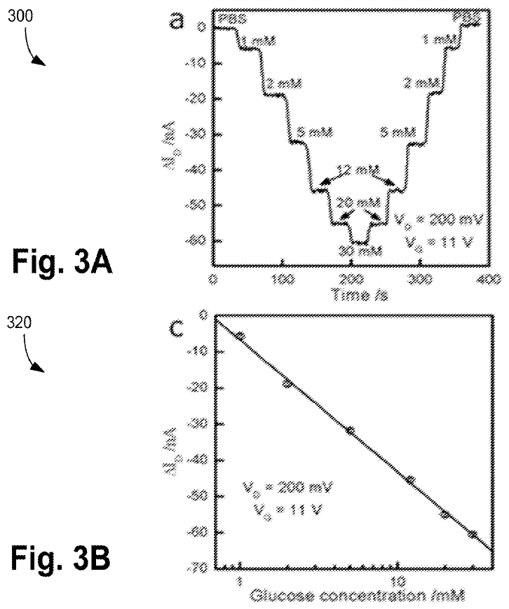

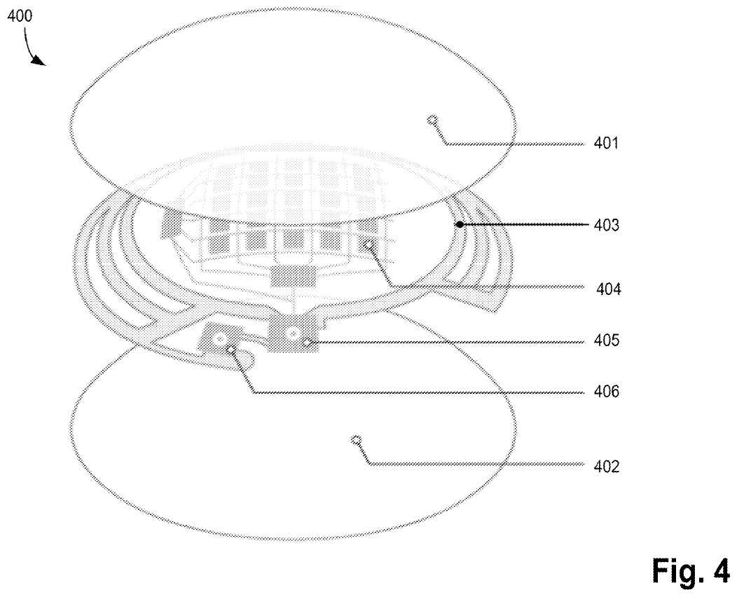

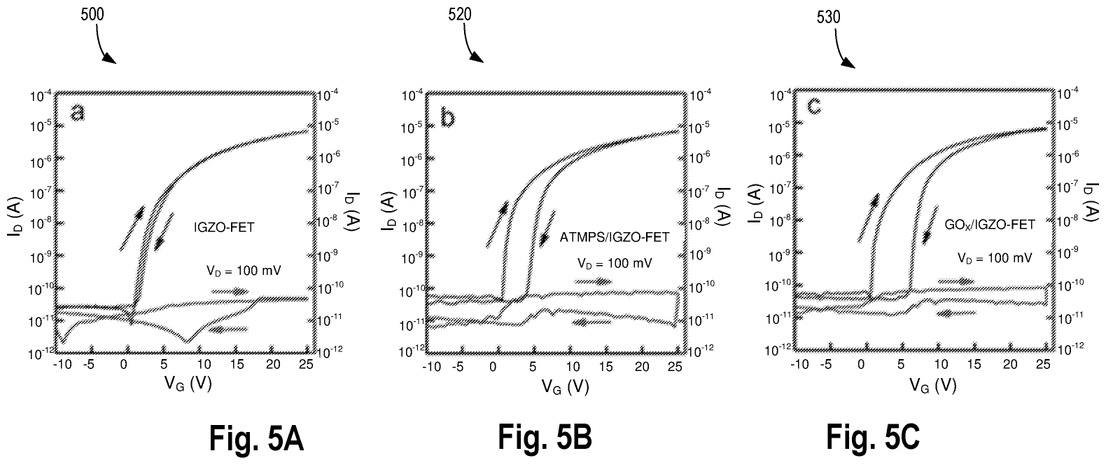

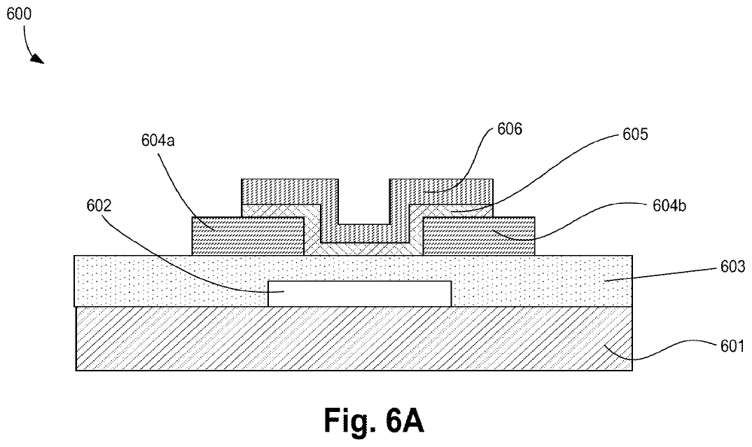

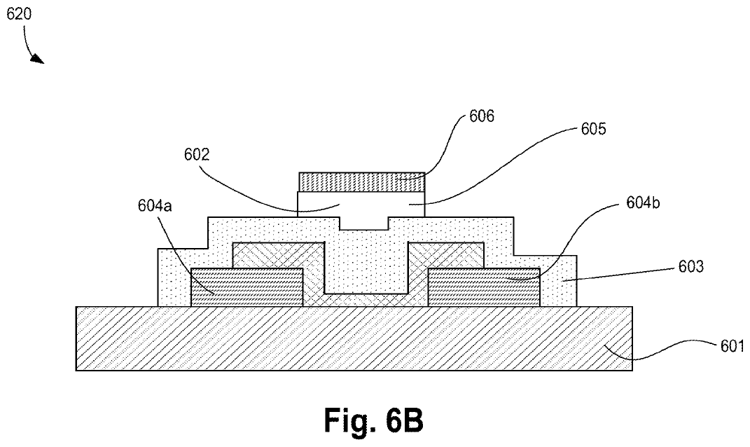

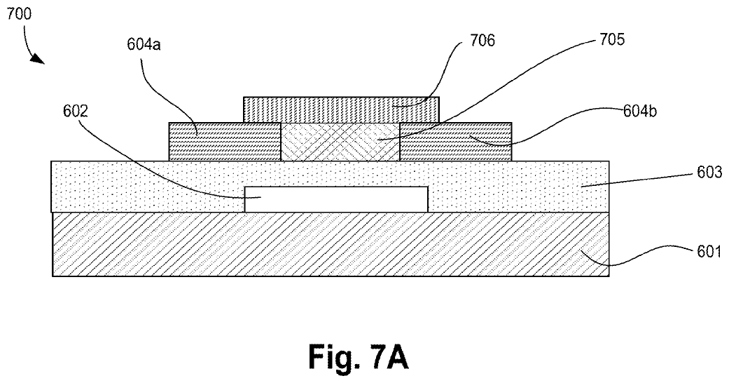

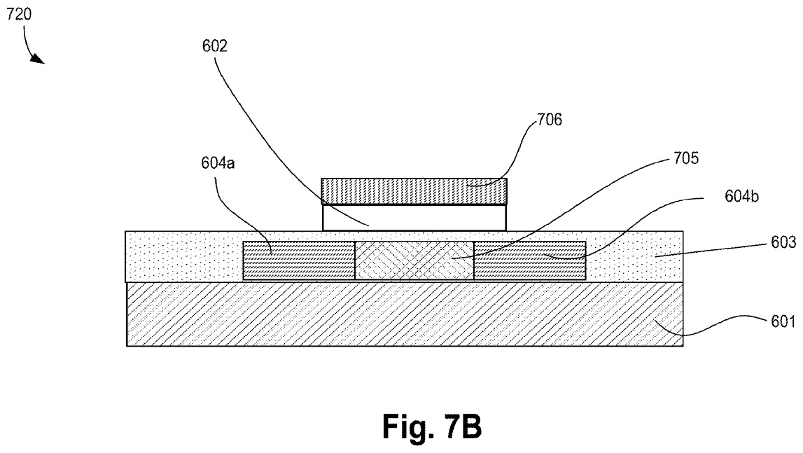

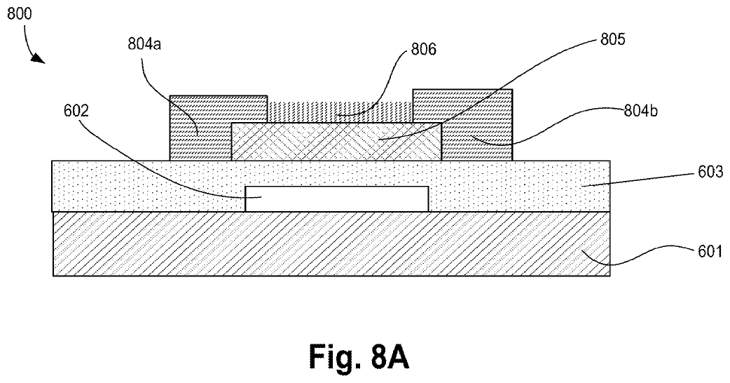

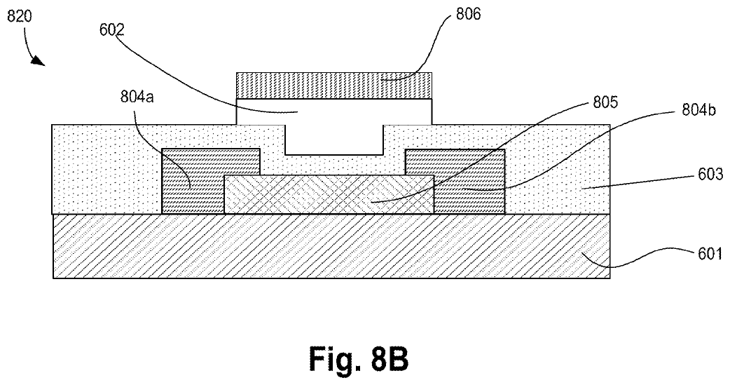

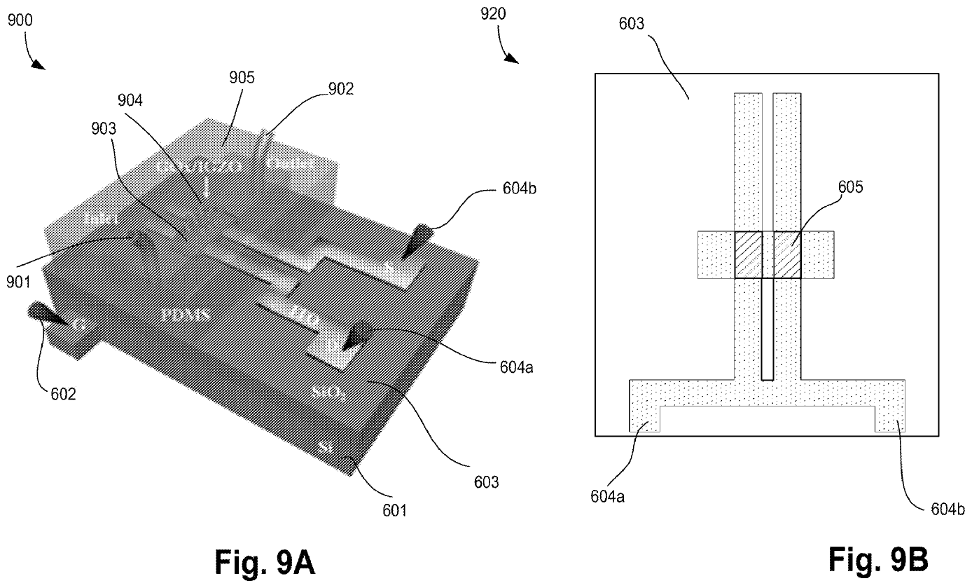

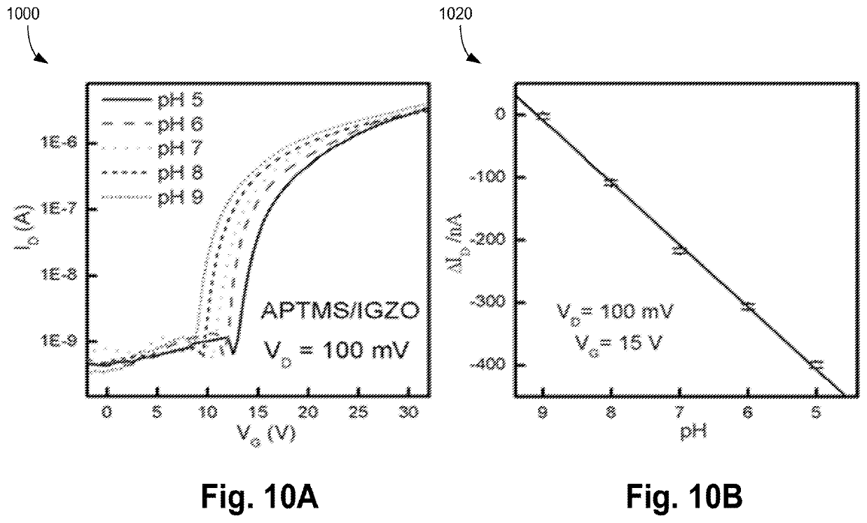

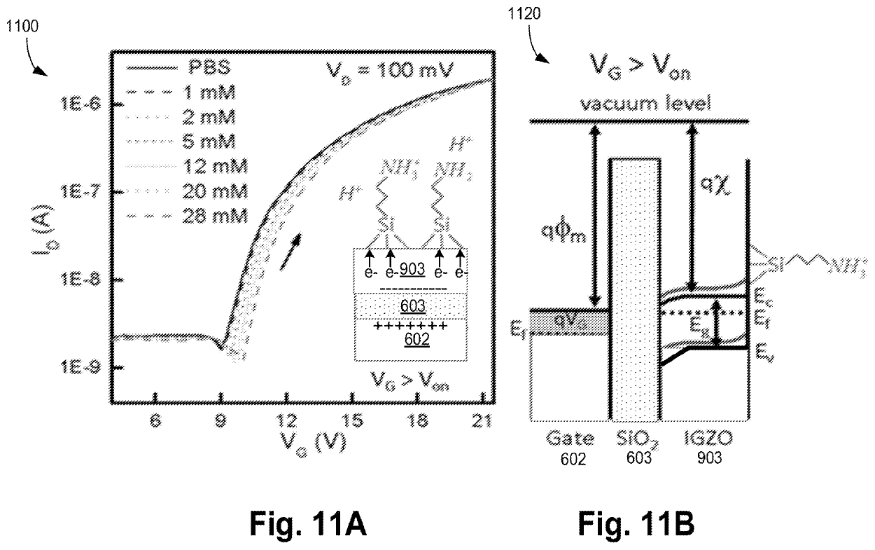

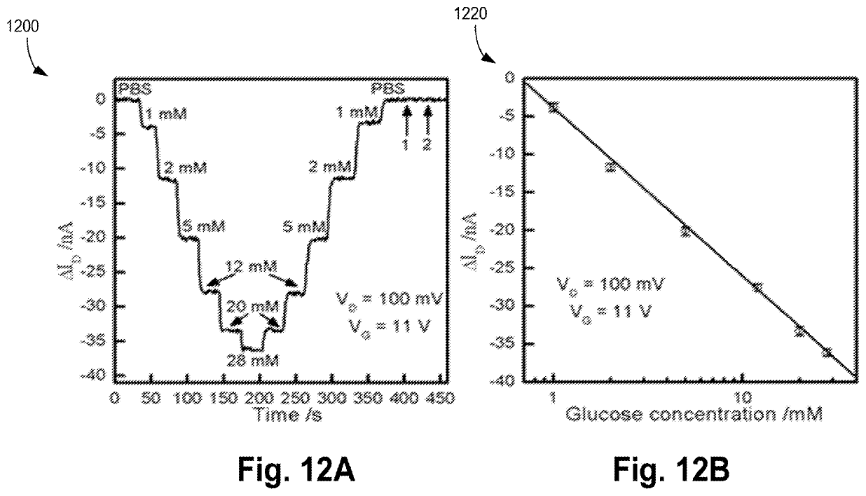

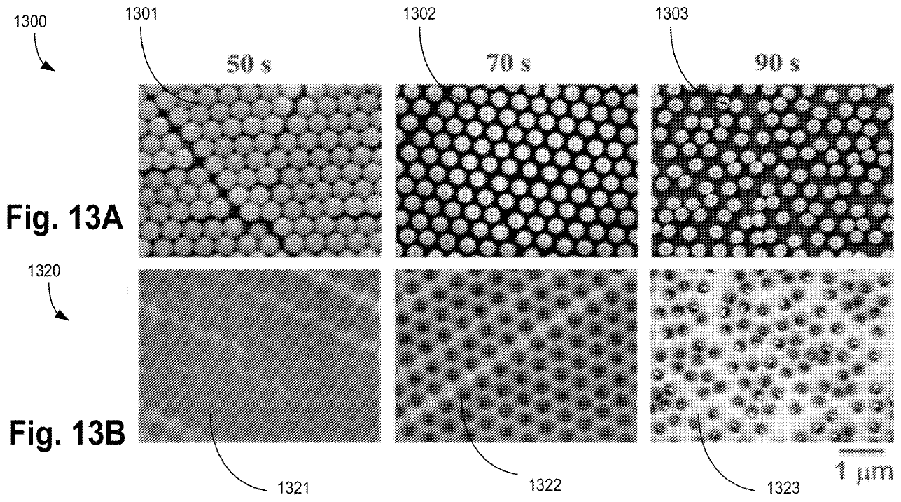

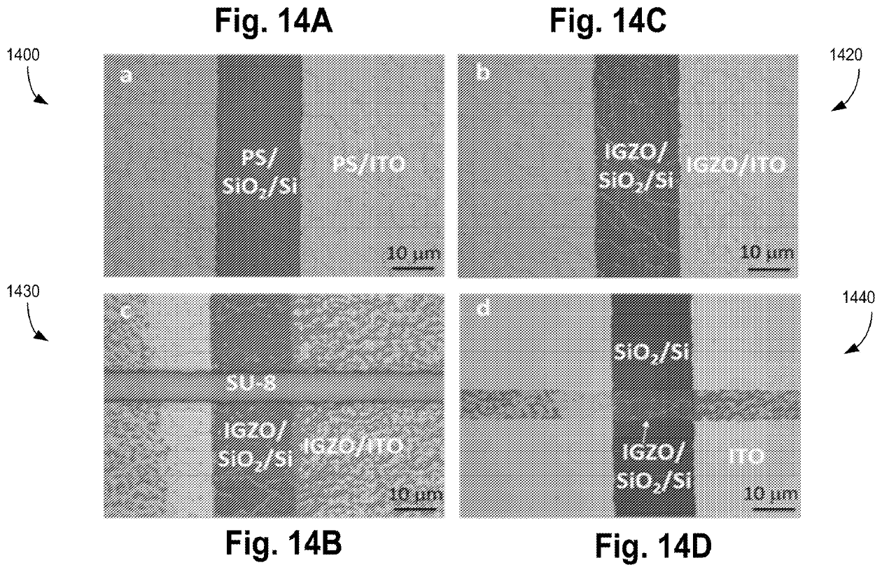

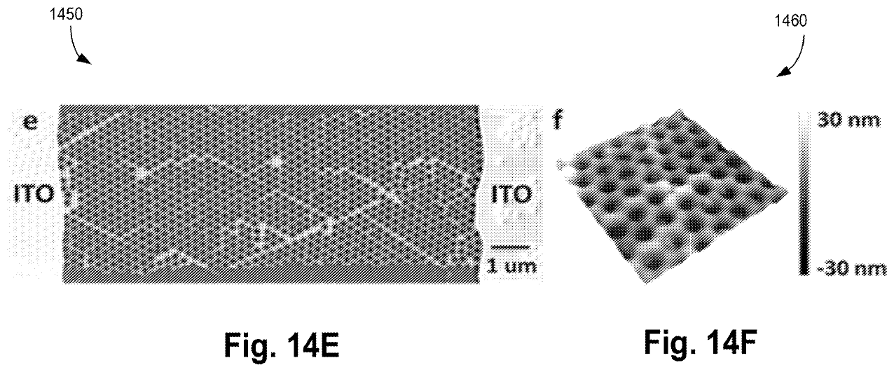

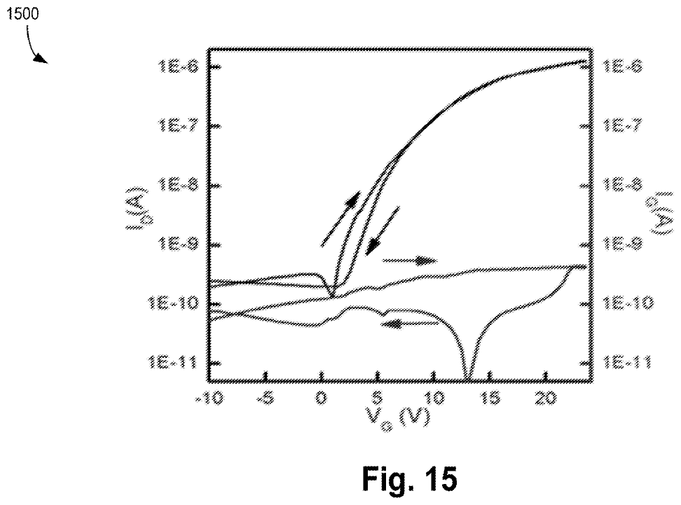

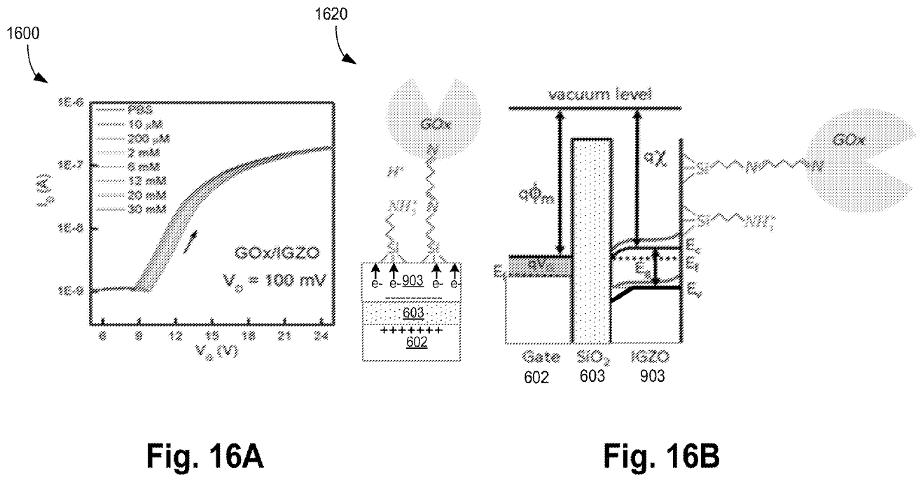

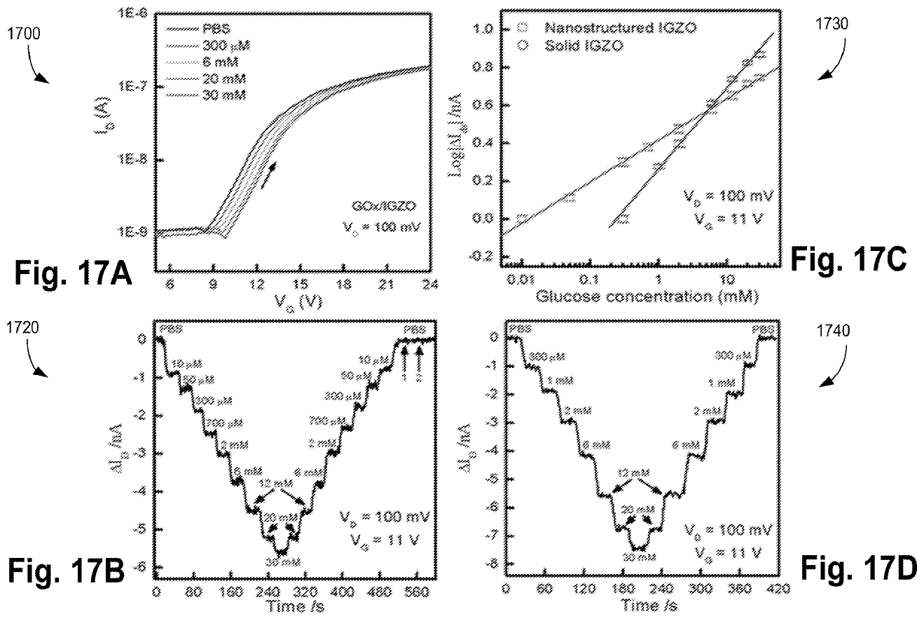

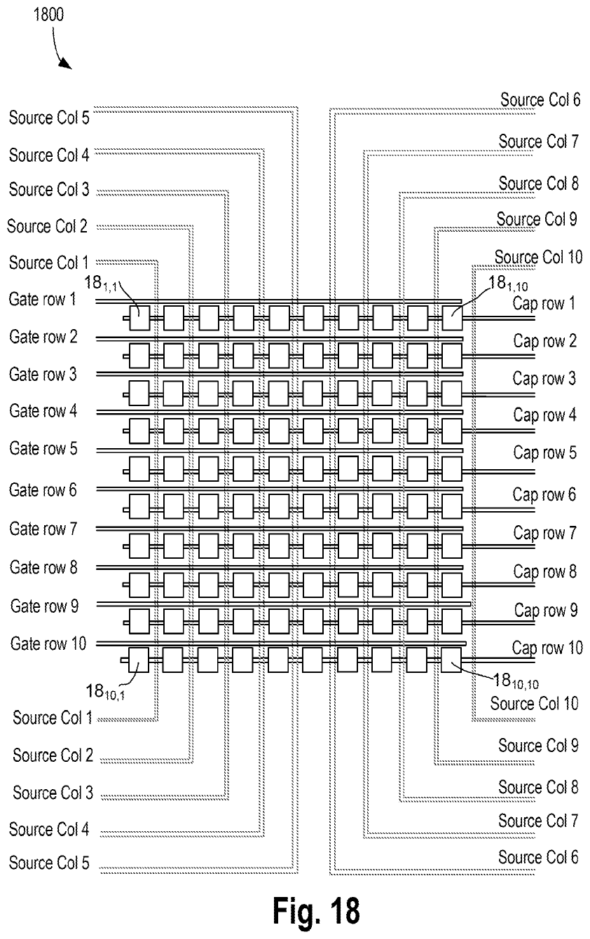

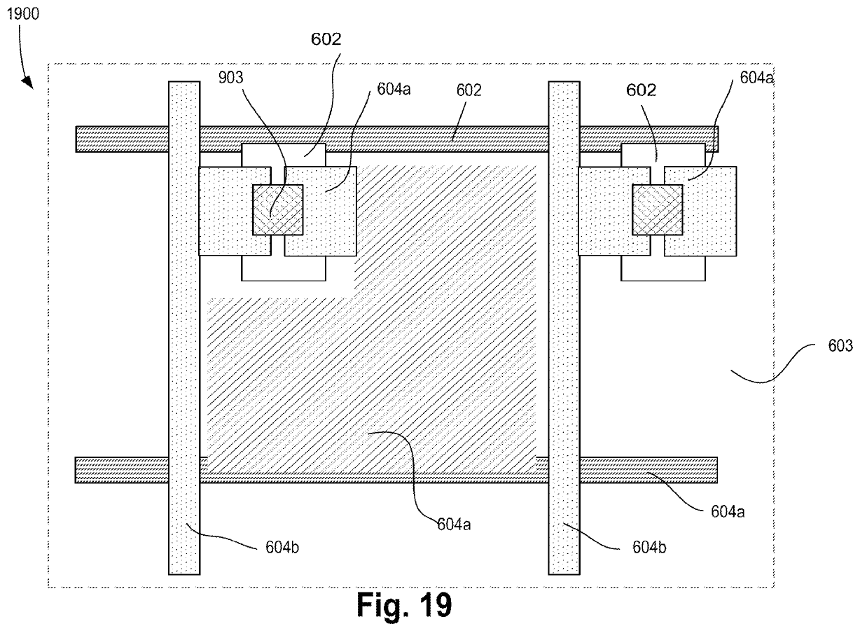

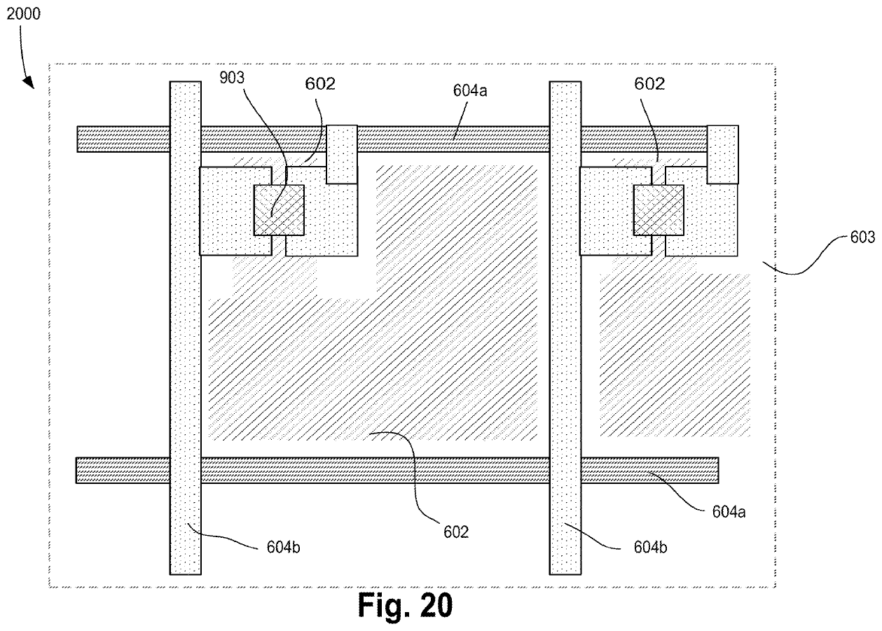

FIG. 2A illustrates a plot showing time course of S/D current change for varying concentrations of glucose as measured using IGZO FET functionalized with GOx, where arrows 1 and 2 indicate addition of 0.13 mM acetaminophen and ascorbic acid, respectively, according to some embodiments.

FIG. 12B illustrates a plot showing S/D current change vs. logarithmic glucose concentrations, according to some embodiments of the disclosure.

FIG. 13A illustrates optical images of polystyrene sphere (PS) monolayer on Sift/Si substrate after oxygen plasma treatment, according to some embodiments of the disclosure.

FIG. 13B illustrates pictures of IGZO nanostructure on Sift/Si substrate after PS liftoff, according to some embodiments of the disclosure.

FIGS. 14A-D illustrate optical images of PS monolayer on ITO S/D by spin coating, nanostructured IGZO film on ITO S/D after PS liftoff, E-jet printed SU-8 wire on nanostructured IGZO film, and final device after IGZO etching, SU-8 developing and oxygen plasma cleaning, respectively, in accordance with some embodiments.

FIGS. 14E-F illustrate an SEM image of nanostructured IGZO wire as channel between ITO S/D electrodes, and AFM image of IGZO closely packed hexagonal nanowires (3×3 μm²), according to some embodiments of the disclosure.

FIG. 15 illustrates a plot showing transfer characteristics of nanostructured IGZO-FET, in accordance with some embodiments.

FIG. 16A illustrates a plot showing IGZO-FET backchannel functionalized with GOx exposed to varying concentrations of glucose, according to some embodiments.

FIG. 16B illustrates a schematic diagram showing the role of positively charged aminosilane groups as an electron acceptor and its impact on band bending at IGZO surface, in accordance with some embodiments.

FIG. 3A illustrates a plot showing transfer characteristics of an IGZO FET backchannel functionalized with GOx exposed to varying concentrations of glucose.

FIG. 17B illustrates a plot showing difference in drain current (AID) versus time for various glucose concentrations as measured using a nanostructured IGZO-FET functionalized with GOx, in accordance with some embodiments, where arrows 1 and 2 indicate the addition of 0.13 mM acetaminophen and ascorbic acid, respectively.

FIG. 17C illustrates a plot showing difference in drain current (AID) versus time for various glucose concentrations as measured using |ΔIds| versus C_{glucose} on a logarithmic scale for solid and nanostructured IGZO-FETs functionalized with GOx, in accordance with some embodiments.

FIG. 17D illustrates a plot showing difference in drain current (AID) versus time for various glucose concentrations as measured using an 8 μm wide solid IGZO-FET functionalized with GOx.

FIG. 18 illustrates a sensing array of IGZO-FET pixels, according to some embodiments of the disclosure.

FIG. 19 illustrates a top view of an IGZO-FET pixel showing the source, drain, and gate terminals, and location of the sensing enzyme on the IGZO, in accordance with some embodiments.

FIG. 20 illustrates a top view of an IGZO-FET pixel showing the source, drain, and gate terminals, and location of the sensing enzyme on the gate, in accordance with some embodiments.

### DETAILED DESCRIPTION

Extremes in blood glucose levels can lead to major health issues. High blood sugar (hyperglycemia) can cause complications such as macrovascular and kidney disease, while low blood sugar (hypoglycemia) can lead to seizures and loss of consciousness. Glucose sensors are a critical component of an artificial pancreas and have been extensively studied during the past several decades. For these applications the sensors need to be sensitive and reliable while measuring glucose concentrations over the normal physiological range (e.g., 2-30 mM in the interstitial fluid and 0.1-0.4 mM in tear fluid).

The recent development of continuous glucose sensors allow real-time monitoring of sugar levels and continuous subcutaneous infusion of insulin/glucagon to assist in maintaining glycemic control. In some embodiments, glucose sensors are integrated as an array of transparent sensors into a contact lens, where such sensors can detect glucose levels from a persons' tears. In some embodiments, these sensors are powered by a capacitive device integrated within the contact lens.

Common amperometric sensors include a Ag/AgCl counter/reference electrode and a platinum working electrode which is coated with a sensing enzyme and a permselective membrane. The disadvantage of the enzyme-based amperometric sensor is the high oxidation potential required on the sensing electrodes for glucose sensing. Potentially other methods may have advantages for glucose sensing, including field-effect transistors (FET) which can be a simple and cost-effective approach. FET-based glucose sensors utilizing boronic acid functionalized carbon nanotubes, as the channel material, have recently been developed. The sensor exhibits high sensitivity and selectivity for glucose in the range of 1 μM-100 mM. An organic electrochemical FET has been fabricated as a glucose sensor, where all the electrodes (source/drain and gate electrodes) and channel materials are made of poly(3,4-ethylenedioxythiphene):poly(styrene sulfonate). The reaction between glucose and the sensing enzyme generates H₂O₂ which can reduce positive poly(3,4-ethylenedioxythiphene) to its neutral state. Therefore, the current change between the source/drain electrodes is proportional to glucose concentration. FET sensors using graphene as the sensing materials have also been demonstrated. By measuring the differential source-drain current, graphene-based sensors can detect glucose levels in the range of medical examination for diabetes diagnostic.

Amorphous indium (In) gallium (Ga) zinc (Zn) oxide (O) (IGZO) field effect transistors (FETs) are used for a wide range of applications. IGZO-FETs are a promising technology that is currently being commercialized in displays. IGZO-FETs have relatively high average electron mobility (e.g., μ_{avg} greater than 10 cm²/Vs) and can be processed at low temperatures that are compatible with flexible transparent substrates. Interest in IGZO-FETs for a range of sensing applications has recently increased in areas including temperature, light sensing, and chemical and biochemical sensors. For example, DNA molecules can be detected through electrostatic interactions with IGZO surface by negatively charged phosphate groups. Organic-capping layers have been used to improve selectivity of IGZO-FETs used as a sensitive gas sensor. Flexible IGZO-FETs, with a compressible dielectric layer, have also been proposed as pressure sensors that can be integrated into contact lenses.

Various embodiments describe functionalized transparent IGZO-FETs that have glucose concentration-dependent changes in the electrical response. Here, the term “transparent” generally refers to a property that is easily seen through and/or can admit passage of light through it. In some embodiments, the positively charged aminosilane groups on the IGZO surface introduce an acceptor-like surface state which can capture electrons from IGZO conduction band and deplete electron carriers in n-type IGZO film below. This leads to decreased drain-source conductance and a more positive V_{ON} with increasing glucose concentration.

In some embodiments, continuous monitoring of drain-source current shows stepwise and fully reversible response to glucose with a short response time. A linear relationship between drain-source current change and logarithmic glucose concentration is observed. Another electrical measurement that can be performed to correlate FET performance to glucose concentrations can include measuring the voltage required to obtain a given current, in accordance with some embodiments. The functionalized IGZO-FET device of various embodiments is effective in minimizing interference from acetaminophen/ascorbic acid. IGZO FETs of various embodiments can be effective for monitoring glucose concentrations in a variety of environments, including fully transparent sensing elements in contact lenses. In some embodiments, these sensing elements are arranged in an array format (e.g., as a matrix having rows and columns of sensing elements).

Some embodiments describe functionalized IGZO-FET back channel surfaces with aminosilane and glucose oxidase enzyme. In some embodiments, the interaction of glucose with the sensing enzyme results in concentration-dependent changes in the electrical response of IGZO-FETs. Various embodiments demonstrate that functionalized IGZO-FETs can be used to sensitively and selectively quantify subtle changes in glucose concentrations in physiological buffers. These results from various embodiments provide insight into a route to develop low-cost transparent biochemical sensors based on the emerging amorphous IGZO (a-IGZO) FET technology.

Various embodiments demonstrate that IGZO FETs can be used as glucose sensors. In some embodiments, highly sensitive IGZO-FETs in aqueous media are fabricated by functionalization of the oxide back channel surface with GOx (glucose oxidase) sensing enzyme. The generated protons from glucose/GOx reactions in the vicinity of pH-sensitive aminosilane groups on IGZO surfaces induce the drain-source current decrease and more positive turn-on voltage in the transfer curve. It is also determined that the drain-source current change is proportional to logarithmic glucose concentrations over the normal range typically found in patients with diabetes. The specific catalysis reaction between GOx enzyme and glucose enable to reduce interference from acetaminophen/ascorbic acid. Various embodiments advance the development of oxide-based FETs for application to glucose biosensors.

Metal oxide and carbon nanomaterials have the potential to make high-performance, transparent, active electronic materials. Transparent thin film transistors are used in simple circuits and transparent displays. In some embodiments, transparent thin film transistors can be combined with sensing circuitries for a range of novel bio-sensing applications. These applications range from simultaneous electrophysiological recordings and neural imaging, integrated bioelectronics on an endoscope, smart contact lenses, and pressure sensors for medical applications and soft robotics, for example. In some embodiments, transparent electrodes are integrated with transparent transistors on flexible transparent substrates, which can then be transferred to a desired substrate. In some embodiments, an array of sensors is formed that can be integrated into new form factors for unique applications.

Various embodiments describe fully transparent biosensors that can be integrated into a transparent substrate. In some embodiments, the fully transparent biosensors are operable to monitor glucose levels for diabetic patients. In some embodiments, the fully transparent biosensors are operable to monitor pH levels. For example, the fully transparent biosensors can monitor uric acid. In some embodiments, the fully transparent biosensors are based on IGZO-FETs that have enzymes attached to the backchannel. The fully transparent biosensors of some embodiments result in very high sensitivities and selectivity of the to-be sensed biomaterial (e.g., drug metabolites), where potential interfering compounds such as acetaminophen and ascorbic acid are completely suppressed.

In some embodiments, certain chemical materials/compounds are integrated to the IGZO-FET sensors to sense a wide variety of chemicals, metabolites, proteins, antibodies and other biomarkers. The fully transparent IGZO-FET sensors of various embodiments can be used to measure pulse, blinking rate, eye movement, sleep abnormalities, etc., which can be correlated with posttraumatic stress disorders. In some embodiments, the fully transparent IGZO-FET sensors are integrated into an active matrix sensing array (e.g., up to 2,500 unique sensors into 1 mm²). As such, a wider range of diseases and treatments can be diagnosed. In some embodiments, fully transparent electronics are developed for biological applications, where the combination of sensing and imaging may improve patient healthcare diagnostics.

In some embodiments, the fully transparent IGZO-FET sensors are directly fabricated on highly curved substrates. In some embodiments, the IGZO FET channel and indium tin oxide (ITO) electrodes is patterned directly on glass tubes (e.g., 2.0 mm diameter) using microcontact printing of self-assembled monolayer (SAM) and wet etching. The fully transparent IGZO FETs results in excellent electronic performance. For example, on/off drain current of approximately 1.3×10⁶ A, average electron mobility greater than 7.4 cm²/Vs, on/off hysteresis of approximately 0.6 V, and gate leakage current of 10⁻¹⁰ A is achieved.

In some embodiments, the back-channel of the IGZO FETs is functionalized with enzymes for selective bio-sensing. In some embodiments, these functionalized IGZO FET based biosensors demonstrate a very high sensitivity to subtle changes in glucose and uric acid concentrations in physiological buffer solutions, where concentrations as low as 1 mM and 50 μM can be readily detected, respectively. These results provide insight into new methodologies for low-cost manufacturing approaches of IGZO FET based biosensors for a range of potential applications, including transparent sensing catheters, flexible active transparent electrode sensing arrays, integration on optical fibers, and on the end of endoscopes.

In some embodiments, patterns are directly generated on curved substrates by laser machining and imprint lithography techniques. In some embodiments, soft lithography, such as micro-contact printing (μCP) is used where complex, three-dimensional (3D) topologies with sub-micrometer-scale features are fabricated on curved surfaces. In some embodiments, patterns are directly generated on curved substrates by patterning functional inks using inkjet, electrohydrodynamic and aerosol printing techniques. These approaches can be used for large-scale manufacturing, for example, for roll-to-roll processes.

μCP of various embodiments takes advantage of the ability of an elastomeric stamp to conform to a non-planar substrate with minimal distortion of the pattern. Self-assembled monolayers (SAMs) often transferred from a patterned stamp onto the surface which results in robust, dense protective SAM layers that can be used as a resist to prevent chemical etching, in accordance with some embodiments. μCP is a convenient, low-cost method to pattern transparent oxide films, and field effect transistors (FETs), in accordance with some embodiments.

One benefit of amorphous IGZO (a-IGZO) for flexible electronics, is that it enables low processing temperatures on flexible, polymeric substrates, while retaining relatively large electron mobility, low operating voltages, and very low off currents. Recently, a-IGZO FETs have been widely studied for use as sensors, including gas detection, temperature, light sensing, pressure sensing in contact lenses, and biochemical sensing. Furthermore, a-IGZO FETs have been demonstrated as an efficient approach to detect glucose levels for diabetes diagnostics, and can potentially work as a critical component of an artificial pancreas. Although a-IGZO FETs on polymer films can be transferred to a range of substrates for sensing or other applications, there may be issues with delamination of the a-IGZO devices from the polymer film or from the polymer film from the substrate.

Various embodiments describe a facile, low-cost methodology to fabricate bottom contact, bottom gate a-IGZO FETs directly on highly curved, transparent substrates by soft lithography. In some embodiments, standard sputter deposition methods are used to deposit high quality IGZO and ITO layers. In some embodiments, μCP is used to pattern the films on glass tubes (e.g., 2.0 mm glass tubes) to form the semiconductor channel and gate/source/drain electrodes, respectively.

The device performance for these a-IGZO FETs is comparable to devices fabricated on planar substrates. In some embodiments, for sensing applications, the back-channel of the IGZO surface is functionalized with sensing enzymes which provide a sensitive response to analyte solutions. In some embodiments, for sensing applications, the gate electrode of the IGZO-FET is functionalized with sensing enzymes which provide a sensitive response to analyte solutions. These results provide insight into low-cost manufacturing methodologies for a-IGZO FETs directly on highly curved surfaces, where applications such as development of a transparent sensing catheter which can combine with the delivery pump in artificial pancreas system, or a range of chemical and/or biological FET sensors integrated onto optical fibers. In some embodiments, μCP is used to pattern films on concave and convex surfaces, where applications can include the development of transparent sensing contact lenses.

In the following description, numerous details are discussed to provide a more thorough explanation of embodiments of the present disclosure. It will be apparent, however, to one skilled in the art, that embodiments of the present disclosure may be practiced without these specific details. In other instances, well-known structures and devices are shown in block diagram form, rather than in detail, in order to avoid obscuring embodiments of the present disclosure.

Note that in the corresponding drawings of the embodiments, signals are represented with lines. Some lines may be thicker, to indicate more constituent signal paths, and/or have arrows at one or more ends, to indicate primary information flow direction. Such indications are not intended to be limiting. Rather, the lines are used in connection with one or more exemplary embodiments to facilitate easier understanding of a circuit or a logical unit. Any represented signal, as dictated by design needs or preferences, may actually comprise one or more signals that may travel in either direction and may be implemented with any suitable type of signal scheme.

The term “device” may generally refer to an apparatus according to the context of the usage of that term. For example, a device may refer to a stack of layers or structures, a single structure or layer, a connection of various structures having active and/or passive elements, etc. Generally, a device is a three-dimensional structure with a plane along the x-y direction and a height along the z direction of an x-y-z Cartesian coordinate system. The plane of the device may also be the plane of an apparatus which comprises the device.

Throughout the specification, and in the claims, the term “connected” means a direct connection, such as electrical, mechanical, or magnetic connection between the things that are connected, without any intermediary devices.

The term “coupled” means a direct or indirect connection, such as a direct electrical, mechanical, or magnetic connection between the things that are connected or an indirect connection, through one or more passive or active intermediary devices.

The term “adjacent” here generally refers to a position of a thing being next to (e.g., immediately next to or close to with one or more things between them) or adjoining another thing (e.g., abutting it).

The term “circuit” or “module” may refer to one or more passive and/or active components that are arranged to cooperate with one another to provide a desired function.

The term “signal” may refer to at least one current signal, voltage signal, magnetic signal, or data/clock signal. The meaning of “a,” “an,” and “the” include plural references. The meaning of “in” includes “in” and “on.”

The term “scaling” generally refers to converting a design (schematic and layout) from one process technology to another process technology and subsequently being reduced in layout area. The term “scaling” generally also refers to downsizing layout and devices within the same technology node. The term “scaling” may also refer to adjusting (e.g., slowing down or speeding up—i.e. scaling down, or scaling up respectively) of a signal frequency relative to another parameter, for example, power supply level.

The terms “substantially,” “close,” “approximately,” “near,” and “about,” generally refer to being within +/−10% of a target value. For example, unless otherwise specified in the explicit context of their use, the terms “substantially equal,” “about equal” and “approximately equal” mean that there is no more than incidental variation between among things so described. In the art, such variation is typically no more than +/−10% of a predetermined target value.

Unless otherwise specified the use of the ordinal adjectives “first,” “second,” and “third,” etc., to describe a common object, merely indicate that different instances of like objects are being referred to, and are not intended to imply that the objects so described must be in a given sequence, either temporally, spatially, in ranking or in any other manner.

For the purposes of the present disclosure, phrases “A and/or B” and “A or B” mean (A), (B), or (A and B). For the purposes of the present disclosure, the phrase “A, B, and/or C” means (A), (B), (C), (A and B), (A and C), (B and C), or (A, B and C).

The terms “left,” “right,” “front,” “back,” to “bottom,” “over,” “under,” and the like in the description and in the claims, if any, are used for descriptive purposes and not necessarily for describing permanent relative positions. For example, the terms “over,” “under,” “front side,” “back side,” “top,” “bottom,” “over,” “under,” and “on” as used herein refer to a relative position of one component, structure, or material with respect to other referenced components, structures or materials within a device, where such physical relationships are noteworthy. These terms are employed herein for descriptive purposes only and predominantly within the context of a device z-axis and therefore may be relative to an orientation of a device. Hence, a first material “over” a second material in the context of a figure provided herein may also be “under” the second material if the device is oriented upside-down relative to the context of the figure provided. In the context of materials, one material disposed over or under another may be directly in contact or may have one or more intervening materials. Moreover, one material disposed between two materials may be directly in contact with the two layers or may have one or more intervening layers. In contrast, a first material “on” a second material is in direct contact with that second material. Similar distinctions are to be made in the context of component assemblies.

The term “between” may be employed in the context of the z-axis, x-axis or y-axis of a device. A material that is between two other materials may be in contact with one or both of those materials, or it may be separated from both of the other two materials by one or more intervening materials. A material “between” two other materials may therefore be in contact with either of the other two materials, or it may be coupled to the other two materials through an intervening material. A device that is between two other devices may be directly connected to one or both of those devices, or it may be separated from both of the other two devices by one or more intervening devices.

Here, multiple non-silicon semiconductor material layers may be stacked within a single transistor structure. The multiple non-silicon semiconductor material layers may include one or more “P-type” layers that are suitable for P-type transistors. The multiple non-silicon semiconductor material layers may further include one or more “N-type” layers that are suitable for N-type transistors. The multiple non-silicon semiconductor material layers may further include one or more intervening layers separating the N-type from the P-type layers. The intervening layers may be at least partially sacrificial, for example to allow one or more of a gate, source, or drain to wrap completely around a channel region of one or more of the N-type and P-type transistors. The multiple non-silicon semiconductor material layers may be fabricated, at least in part, with self-aligned techniques such that a stacked device may include both N-type and P-type transistor with a footprint of a single transistor.

It is pointed out that those elements of the figures having the same reference numbers (or names) as the elements of any other figure can operate or function in any manner similar to that described, but are not limited to such.

FIGS. 1A-D illustrate a process (100, 120, 130, and 140, respectively) of micro-contact printing on a curved substrate, according to some embodiments. The process applies a polydimethylsiloxane (PDMS) pad 101 on a curved substrate having metal oxide 102. A curved substrate is passed over a patterned elastomeric stamp 103 that has a self-assembled monolayer resist material which is transferred to the metal oxide 102. In some embodiments, high-performance a-IGZO FETs on curved substrates are fabricated using micro-contact printing (μCP) to pattern sputter deposited IGZO and ITO films. In some embodiments, these films are patterned without visible defects using PDMS stamp 101 to transfer octadecylphosphonic acid (OPDA), followed by oxalic acid etching as illustrated by FIGS. 1C-D. In some embodiments, functional inks can be directly transferred to the curved substrate.

In some embodiments, the patterned elastomeric stamp 103 is inked with a self-assembled monolayer (SAM) of octadecylphosphonic acid (ODPA). Since the elastomeric stamp 103 can conform to the nonplanar substrate, with minimal distortion of its pattern, the SAMs are directly transferred from the elastomeric stamp 103 to the oxide film 102 by rolling the tube across the stamp surface, according to some embodiments. In some embodiments, the SAMs 104 are used as a chemical protection layer during wet etching where the uncovered metal oxide can be selectively etched in oxalic acid solution.

In some embodiments, Scanning Electron Microscope (SEM) images indicate well-defined step edges from one layer to another. The a-IGZO FETs fabricated by this convenient and low-cost route have excellent electrical characteristics. In some embodiments, the immobilized sensing enzyme on a-IGZO back-channel provides stepwise and fully reversible response to analyte solutions with a short response time. These results suggest that a-IGZO FETs may provide a means to integrate fully transparent, highly-sensitive sensors onto catheters and optical fibers.

FIG. 1E illustrates an optical image 150 of an Amorphous indium (In) gallium (Ga) zinc (Zn) oxide (0) (IGZO) field effect transistors (FET), according to some embodiments. Image 150 illustrates the drain 151, source 152, and the IGZO material 153 over metal oxide dielectric 102 (e.g., Al₂O₃), which is located on top of an ITO gate electrode (not shown).

FIG. 1F illustrates a schematic 160 of an IGZO FET structure on a curved substrate, according to some embodiments. The IGZO FET in this example is formed over a curved substrate having a gate terminal 163 and a metal oxide dielectric 102 (e.g., Al₂O₃). Drain 161 and Source 162 terminals are formed that coupled to the IGZO material 164. In some embodiments, the gate terminal 163 is formed on the substrate 165.

FIG. 1G illustrates a Scanning Electron Microscopy (SEM) image 170 of a step edge between an Indium Tin Oxide (ITO) and substrate and between an IGZO/ITO and IGZO, according to some embodiments. In some embodiments, a bottom contact, bottom gate structure is used due to the relative etch selectivity in oxalic acid (e.g., much higher etch rate for IGZO compared to ITO). SEM is used here to characterize the step edge profiles of patterned ITO source and drain as shown in FIG. 1G. FIG. 1G also shows the smooth and defect-free transition from the IGZO layer to the region that contains both IGZO and ITO and the region that has the Al₂O₃ gate dielectric, in accordance with some embodiments.

In some embodiments, μCP ODPA transferred ink is continuous and provides excellent etch resistance to oxalic acid. In some embodiments, the step edge between ITO source/drain and the Al₂O₃ dielectric layer 103 may not show any resolution loss or visible defects compared to the master or the PDMS stamp 101. In some embodiments, IGZO deposition and μCP yields good quality patterning of the channel material, which is used to obtain uniform device performance for IGZO FETs.

FIGS. 2A-B illustrate plots 200 and 220 showing output characteristics and transfer characteristics of IGZO-FET on curved substrate fabricated by μCP with W/L ratio of 100 μm/20 μm, respectively, according to some embodiments. The electrical properties of the fabricated IGZO FETs are measured and the output characteristics are shown in FIG. 2A, where the drain current (I_{D}) 201, 202, and 203 is measured while sweeping the drain voltage (V_{D}) for several gate voltages (V_{G}).

A typical accumulation mode n-channel transistor behavior is observed where a linear regime corresponds to the field-effect current modulation and a saturation regime is observed at high V_{G} (gate voltage). In some embodiments, no current crowding is observed for these devices suggesting good source/drain (S/D) contact to the IGZO film. Representative transfer characteristics are shown in FIG. 2B, where the I_{D} (drain current) is measured while sweeping V_{G} from −6 V up to 26 V and back down to −6 V with a constant drain voltage V_{D} (1 V) as shown by waveforms 221 and 222. Also shown in FIG. 2B is the gate leakage (see waveforms 223 and 224) for the a-IGZO FET.

Table I shows the average electron mobility (μ_{avg}), turn-on voltage (V_{ON}), drain current on-to-off ratio (I_{ON}/I_{OFF}), and hysteresis.

TABLE 1

| IGZO-TFT | μ_{avg} (cm²/Vs) | Vₒₙ (V) | Iₒₙ/I_{off} | Hysteresis (V) |
|---|---|---|---|---|
| μCP | 7.4 | 3.2 | 1.3 × 10⁶ | 0.6 |

In this example, the small hysteresis observed between the up and down sweeps indicate that traps can stay in equilibrium with the sweep rate of V_{G}=0.2 V/s. A high I_{ON}/I_{OFF} ratio ˜10⁶ and high μ_{avg} greater than 7 cm²/Vs are obtained, while the gate leakage current (I_{G}) is typically less than nA/cm² in this example. These results indicate that μCP patterned metal oxides on glass tubes are robust for device integration, have good semiconductor/source and drain interfaces, and have high-quality dielectric/channel interface, in accordance with some embodiments.

Functionalized a-IGZO FETs in response to varying concentration of glucose and uric acid are tested, in accordance with some embodiments. FIGS. 4A-B illustrate plots 300 and 320 showing drain current (I_{D}) versus time for various glucose concentrations as measured using functionalized IGZO-FET on curved substrate, and difference in drain current (AID) versus glucose concentration on a logarithmic scale, according to some embodiments. In some embodiments, glucose/uric acid is delivered to the device in a phosphate buffered saline (PBS) solution using a PDMS well which defines the contact area of the analyte solution to the back channel of the a-IGZO FET. The reaction between the immobilized enzyme and analyte decrease the pH at a-IGZO/electrolyte interface through proton dissociation. The generated protons (lower pH) in the vicinity of pH-sensitive aminosilane groups induce protonation of —NH₂ to —NH₃⁺ on the silanized IGZO surface, in accordance with some embodiments.

The accumulated electrons close to the IGZO backchannel surface are depleted by this acceptor-like state, leading to an increase of the surface band bending. This results in a decrease in conductance and uses a more positive V_{G} to turn on the device. The enzyme functionalized a-IGZO FET is tested for device stability in PBS with a fixed gate voltage, V_{G}=11 V. The change in I_{D} (AID), compared to the PBS solution with no analyte, remains constant for the entire time (e.g., greater than 1000 seconds) indicating excellent stability for these sensors. Continuous monitoring is conducted by measuring I_{D} with V_{G} set to 11 V, which is the maximum in sub-threshold slope for the a-IGZO FET in PBS solution.

The AID (change in drain current) decreases (increases) in a stepwise fashion as C_{glucose}/increases (decreases). AID is found to be fully reversible for increasing and/or decreasing analyte concentrations, in accordance with some embodiments. Inspection of the data indicates that the response time for sensing is less than approximately 10 seconds. The AID is plotted versus C_{glucose} in FIG. 3B and a linear relationship is obtained using a semi-log plot (coefficient of determination R²=0.999) for all tested concentrations.

These results indicate that IGZO FET based biosensors can be directly integrated on high curvature surfaces, in accordance with some embodiments. In some embodiments, functionalizing the IGZO back channel with immobilized enzymes is an effective method to produce sensitive and selective biosensors. In some embodiments, incorporating glucose sensors directly on catheters allows for development of an artificial pancreas for diabetes patients. Other potential applications include the integration of IGZO FET biosensors on optical fibers to increase sensing functionality.

FIG. 4 illustrates a three dimensional view of an apparatus 400 comprising an IGZO FET based biosensor integrated in contact lens, according to some embodiments.

In some embodiments, apparatus 400 comprises a top lens layer 401, a bottom lens layer 402, an array of transparent IGZO FET based biosensors (e.g., the transparent active sensing array) 404, invisible antenna 403, transparent controller 405, and a transparent capacitor 406. Here the term array generally refers to two or more elements which may be positioned as a matrix, for example. In some embodiments, the array of transparent IGZO FET based biosensors 404 are spread on a curved transparent substrate (e.g., polymer substrate) formed according to the various embodiments described here. For example, a micro-concave printing stamp or micro-contact printing (e.g., μCP stamp) is used to form the shape of a transparent substrate and to produce patterned film on the curved transparent substrate.

In some embodiments, the array of transparent IGZO FET based biosensors 404 occupy a region which is in direct line of sight through the top 401 and bottom 402 lens layers. In some embodiments, the transparent IGZO FET based biosensors of the array 404 are uniformly spaced through the entire curved transparent substrate. In some embodiments, the transparent IGZO FET based biosensors of the array 404 are positioned over the pupil region of an eye. For example, the transparent IGZO FET based biosensors 404 are located near the center region of the polymer substrate (e.g., near the center region of the lens).

In some embodiments, an invisible antenna array 403 is also included such that the antennas 403 are positioned between the gaps amid the transparent a-IGZO FET based biosensors of the array 404. In some embodiments, the antenna 403 is an invisible antenna and comprises one or more of: graphene, carbon nanotubes, silver nanowires, and/or copper nanowires which can be transparent and highly conductive.

In some embodiments, the antenna 403 comprises copper which can be configured as narrow and/or tall and positioned along the edges to avoid limiting line of sight or vision through the lens. In some embodiments, the antenna elements 403 are placed in their position using copper electroplating or graphene electrophoretic deposition. For example, the transparent polymer substrate is embossed in the shape of the antenna, and then the embossed region is filled with copper or graphene.

In some embodiments, the horizontal and vertical lines within and around the array of antennas 403 are gate, source, and drain electrodes. For example, the horizontal lines are gate electrodes while the vertical lines are source electrodes. In some embodiments, all transparent a-IGZO FET based biosensors 404 have the same enzyme to check for a particular kind of biomaterial. In some embodiments, each row or column of the array of transparent IGZO FET based biosensors 404 has the same enzymes to improve accuracy of the measurements. In some embodiments, each row or column of the array of transparent IGZO FET based biosensors 404 has the same enzymes where some are deactivated. In some embodiments, each row or column of the array of transparent a-IGZO FET based biosensors 404 has different enzymes. As such, the array of transparent a-IGZO FET based biosensors 404 can sense different biomaterials instead of just one kind of biomaterial. In some embodiments, the enzyme of interest is attached to the transparent a-IGZO FET. In some embodiments, the enzyme of interest is printed to the gate of the a-IGZO FET.

In some embodiments, power is provided to the controller using a capacitor 406 that can be charged remotely. In some embodiments, power is provided to the controller using a battery 406 that can be charged remotely. For example, RF (radio-frequency) techniques can be used to charge the capacitor/battery that provides the necessary power to the controller to sense the source/drain currents and/or to bias the gates of the array of transparent IGZO FET based biosensors 404. In some embodiments, transparent material is used for forming the controller 405 and capacitor 406. As such, the vision or sight is not disturbed or limited in any way, in accordance with some embodiments. In some embodiments, signal lines and/or power lines are routed along the periphery of the concave substrate to avoid any limitation to vision or sight through the top and bottom lens. In some embodiments, the signal lines and/or power lines are made of transparent material (e.g., graphene).

The following section describes the fabrication of the μCP stamp. While various embodiments describe pre-forming the polymer substrate into a concave shape, forming into other shapes is also contemplated by the various shape molding techniques described here. In some embodiments, an epoxy-based negative photoresist (SU-8) is spin-coated onto a clean silicon wafer. In some embodiments, the photomask is aligned in close contact with the wafer and an ultra-violet light source (e.g., model 100UV30S1, Karlsus Inc.) with wavelength of approximately 360 nm used to expose the photoresist. In some embodiments, a developer solution is used to remove unexposed regions of SU-8 from the substrate. The remaining SU-8 pattern on the silicon wafer (the master) may have a depth of approximately 50 μm as measured by profilometry (KLA-Tencor Alpha-Step 500). In some embodiments, Tridecaflouro-tetrahydrooctyl-trichlorosilane TFOCS is deposited as a monolayer on the master through siloxane bonding by placing the master in (TFOCS) vapor for a predetermined time (e.g., 30 minutes). In some embodiments, liquid pre-polymer PDMS with curing agent (e.g., 5:1 weight ratio) is cast onto the master followed by degassing for a predetermined time (e.g., 30 min) in vacuum. In some embodiments, the PDMS is cured in an oven at a certain temperature for a certain duration (e.g., 70° C. for 7 hours) and then a fresh PDMS stamp 103 with features opposite to the master is fabricated by peeling the PDMS stamp from the master substrate.

The following section describes the fabrication of the device. μCP FET test structures can be fabricated as follows, in accordance with some embodiments. In some embodiments, a glass tube (e.g., outside-diameter (OD) of approximately 2 mm) is used as the substrate (e.g., inner region of 102). In some embodiments, ITO films 102 (e.g., 160 nm thick, measured by ellipsometry) are deposited using RF magnetron sputter deposition with a, for example, 3 inch ITO target (e.g., composition: In₂O₃:SnO₂=90:10 wt %), 120 W RF power, approximately 4 mTorr chamber pressure, and 20 sccm flow rate of Ar. In some embodiments, before patterning the gate electrode by μCP, the ITO films are treated by UV-ozone for 15 min (e.g., PSD standard, Novascan).

In some embodiments, an ODPA SAM is formed on the PDMS stamp by immersion in 5 mM ODPA/isopropyl alcohol for a certain time (e.g., 5 min). In some embodiments, the SAM is transferred from the PDMS stamp 103 to oxide film 102 by rolling the tube over the stamp 103. In some embodiments, vertical and/or horizontal lines are then formed on the substrate. These are the electrodes for the array of transparent a-IGZO FET based biosensors.

In some embodiments, after SAM transfer, the unprotected ITO is etched at a certain rate (e.g., rate of 10 nm/min) in 50 mM aqueous oxalic acid solution, with mild agitation. In some embodiments, to remove the ODPA ink, the ITO surface is rinsed with 2-propanol followed by oxygen plasma cleaning for 5 minutes at 50 W (e.g., PE-100, Plasma Etch, Inc.). In some embodiments, the ITO films are then annealed at 300° C. for one hour to increase the crystallinity of the ITO films, which significantly reduces the etch rate to less than 1 nm/min. This ensures the ITO pattern will be preserved during the following etching steps. In some embodiments, the annealing also increases the ITO electrical conductivity, and improves the films transparency.

In some embodiments, Al₂O₃(e.g., 50 nm thick) is deposited as the gate dielectric by any suitable means (e.g., atomic layer deposition (ALD)). In some embodiments, ALD is performed at 200° C. in a Picosun SUNALE R-200 reactor using alternating N₂-purge-separated pulses of O₂ and trimethylaluminum. In some embodiments, the deposition rate is approximately 0.10 nm/cycle. In some embodiments, the second ITO film (100 nm thick, measured by ellipsometry) is deposited and μCP patterned on the substrate as S/D (Source/Drain) electrodes.

In some embodiments, amorphous IGZO films (e.g., approximately 50 nm thick) are deposited by sputter deposition with a 3 inch IGZO sputter target (e.g., molar composition: In₂O₃:Ga₂O₃:ZnO), 100 W RF power, approximately 4 mTorr chamber pressure, and 20 sccm flow rate with a 1:19 (O₂:Ar) ratio. In some embodiments, IGZO active layers are patterned subsequently, using the same procedures as the ITO films with an etch rate of 40 nm/min in 50 mM aqueous oxalic acid solution. In some embodiments, the fabricated FETs are annealed in air at 300° C. to improve the device performance, and the resulting FETs have a width/length (W/L) ratio of 100 μm/20 μm.

The following section describes the surface functionalization. In some embodiments, the IGZO surface is cleaned in oxygen plasma (e.g., 50 W power for 2 mins) to remove contaminants. In some embodiments, it is immediately soaked in 1% ethanol solution of aminopropyltrimethoxysilane (APTMS) for 2 hours, rinsed with ethanol and then dried with flowing nitrogen. In some embodiments, the ATPMS-IGZO film is then immersed in 20 mM glutaraldehyde (GA) in PBS solution for 2 hours. GA acts as the cross-linker molecule to immobilize the sensing enzyme. In some embodiments, the device is transferred and kept in 10 g/L glucose oxidase (GOx) or urease in PBS for 2 hours. In some embodiments, the sample is rinsed with water and then dried with flowing nitrogen prior to electrical measurements.

The following section describes the experimental test process (e.g., glucose detection). In some embodiments, all IGZO-FET electrical measurements are performed in the dark at room temperature using an Agilent 4155C precision semiconductor parameter analyzer. In some embodiments, forward (low voltage to high voltage) and backward (high voltage to low voltage) sweep drain-to-source current versus gate voltage (I_{D}-V_{G}) transfer curves are measured with the drain voltage (V_{D}) set to 1 V at a V_{G} sweep step of 0.2 V and a sweep rate of V_{G}=0.2 V·s⁻¹. In some embodiments, a PDMS well is attached to the top of the exposed IGZO channel. In some embodiments, phosphate buffer solutions for each analyte (100 μl) are introduced into the PDMS well (volume approx. 0.25 μl) using a syringe.

While some embodiments are described with reference to glucose sensing, the embodiments can be extended to sensing other types of enzymes and chemicals. For example, various embodiments can be used to sense or detect deoxyribonucleic acid (DNA), ribonucleic acid (RNA) and other acids, proteins, lipids, carbohydrates, etc.

For purposes of explaining the various embodiments, source of various materials is provided. However, the embodiments are not limited to these sources.

Glucose is obtained from Alfa Aesar. HCl, NaCl, KCl, NaH₂PO₄, Na₂HPO₄ are acquired from Macron. Aminopropyltrimethoxysilane, acetaminophen and ascorbic acid are from Sigma-Aldrich. Glutaraldehyde is from Electron Microscopy Sciences. Glucose oxidase is obtained from Amresco. IGZO and ITO targets are from AJA International Inc. and Kurt J. Lesker Inc., respectively. The photoresist S1818 is from Microchem. Sylgard 184 PDMS is from Dow Corning. Milli-Q water (18.2 MΩ cm) is used in all sample preparation.

FIGS. 5A-C illustrate plots 500, 520, and 530, respectively, showing transfer characteristics of IGZO-FET test structures that are fabricated using a heavily p-doped Si substrate as the gate and thermally grown Sift (e.g., 100 nm thick) as the gate dielectric, according to some embodiments. ITO films (e.g., 160 nm thick, measured by ellipsometry) are deposited on Si/SiO₂ substrate using RF magnetron sputter deposition with a 3 inch ITO target (e.g., composition: In₂O₃:SnO₂=90:10 wt %), 120 W RF power, approximately 4 mTorr chamber pressure, and 20 sccm flow rate of Ar. In some embodiments, source and drain electrodes are patterned using photolithography and etched in HCl (e.g., 1:20 in DI) giving a W/L ratio of, for example, 100 μm/20 μm. In some embodiments, the ITO films are then annealed at, for example, 300° C. for one hour to increase their resistance to HCl etch, increase their electrical conductivity, and improve their transparency.

In some embodiments, Amorphous IGZO films (e.g., approximately 50 nm thick) are deposited by sputter deposition with, for example, a 3 inch IGZO sputter target (e.g., molar composition: In₂O₃:Ga₂O₃:ZnO), 100 W RF power, approximately 4 mTorr chamber pressure, and 20 sccm flow rate with a 1:19 (O₂:Ar) ratio.

In some embodiments, IGZO channel is patterned on top of ITO source and drain (S/D) regions. In some embodiments, the etching solution is diluted HCl in DI (1:200). In some embodiments, the fabricated IGZO-FETs (e.g., transparent FETs) are subsequently annealed in air at, for example, 300° C. to improve the device performance.

FIGS. 6A, 7A, and 8A illustrate cross-sections of various embodiments 600, 700, and 800 of bottom-gated IGZO-FET structures, respectively, according to some embodiments. The cross-section of FIG. 6A illustrates substrate 601, gate 602 (e.g., 102), dielectric 603, source and drain regions 604a/b, respectively, active layer 605, and sensing layer 606. In this embodiment, the active layer 605 is adjacent to dielectric 603 and partially wraps around the source and drain regions 604a/b. In some embodiments, the sensing layer 606 conforms to the pattern of the active layer 605 and is adjacent to the active layer 605.

The cross-section of FIG. 7A illustrates substrate 601, gate 602 (e.g., 102), dielectric 603, source and drain regions 604a/b, respectively, active layer 705, and sensing layer 706. In this embodiment, the active layer 705 is adjacent to dielectric 603 and between the source and drain regions 604a/b. In some embodiments, the sensing layer 706 is fabricated over the active layer 705 such that portion of the sensing layer is adjacent to the source and drain regions 604a/b.

The cross-section of FIG. 8A illustrates substrate 601, gate 602 (e.g., 102), dielectric 603, source and drain regions 804a/b, respectively, active layer 805, and sensing layer 806. In this embodiment, the active layer 805 is adjacent to dielectric 603 and between the source and drain regions 804a/b, while source and drain regions 804a/b are partially over the active layer 805. In some embodiments, the sensing layer 806 is fabricated over the active layer 805 such the sensing layer 806 is between the source and drain regions 804a/b.

In some embodiments, the substrate 601 is formed of one or more of: polyethylene terephthalate (PET), polyethylene naphthalate (PEN), polycarbonate (PC), polyimide (PI), paralene, hydrogels, cellulose based substrates, collagen based substrates, glass, or other similar materials.

In some embodiments, the gate 602 is formed of one or more of: transparent conducting oxides, including indium tin oxide (ITO), doped ZnO, (e.g., Al-, In-, Ga-doped, etc.), doped SnO₂ (e.g., F-, Sb-doped, etc.), zinc indium oxide (ZIO), zinc tin oxide (ZTO); Conducting nanomaterials, metal nanowires (e.g., Ag, Au, Cu, etc.), carbon nanotubes, graphene; Organic conductors, poly(3,4-ethylenedioxythiophene) (PEDOT), PDOT: poly(styrene sulfonate) (PSS); or Structured metal arrays.

In some embodiments, the dielectric 603 is formed of one or more of: SiO₂, Al₂O₃, ZrO₂, HfO₂, Ta₂O₅, SiNx, polyimide, polyvinyl phenol, self-assembled dielectrics, self-assembled multilayer dielectrics.

In some embodiments, the gate 602, and source/drain regions 604a/b (704a/b or 804a/b) are comprises one or more of: transparent conducting oxides, conducting nanomaterials, organic conductors, polymers, or structured metal arrays. In some embodiments, the transparent conducting oxides includes at least one or more of: In, Sn, O, or Zn. In some embodiments, the transparent conducting oxides includes at least one of: indium tin oxide (ITO), doped ZnO (e.g., Al-, In-, Ga-doped, etc.), doped SnO₂ (e.g., F-, Sb-doped, etc.), zinc indium oxide (ZIO), or zinc tin oxide (ZTO). In some embodiments, the conducting nanomaterials include at least one of: metal nanowires, carbon nanotubes, or graphene. In some embodiments, the metal nanowires include one of: Ag, Au, or Cu. In some embodiments, the organic conductors include one of: poly(3,4-ethylenedioxythiophene) (PEDOT), PDOT, or poly styrene sulfonate (PSS).

In some embodiments, the active layer 605 (705 or 805) is formed of one or more of: In_{w}GaₓZn_{y}O_{z}; SnₓZn_{y}O_{z}; InₓZn_{y}O_{z}; InₓGa_{y}O_{z}; or In_{w}SnₓZn_{y}O_{z}, where the subscripts are the relative composition of the indicated elements. In some embodiments, the active layer 605 comprises nanostructures. In some embodiments, the nanostructures comprise Indium-Gallium-Zinc-Oxide (IGZO) nanostructures. In some embodiments, the nanostructures are one of: hexagonal nanowires, parallel lines, square grids, diamond grids, or spiral structures.

In some embodiments, the sensing layer 606 (706 or 806) is formed of one or more of: glucose oxidase, urease, invertase, mutarotase, maltase, alcohol dehydrogenase, aldehyde dehydrogenase, cortisol specific monoclonal antibody, anti-testosterone monoclonal antibody, or peroxidase.

In some embodiments, Surface Functionalization of IGZO-FET is performed as follows. In some embodiments, the IGZO surface is cleaned in oxygen plasma (e.g., 50 W power for 2 mins) to remove contaminants. In some embodiments, the IGZO surface is immediately soaked in 1% ethanol solution of aminopropyltrimethoxysilane (APTMS, H₂N(CH₂)₃Si(OCH₃)₃) for some time (e.g., 2 hours), rinsed with ethanol and then dried with flowing nitrogen. In some embodiments, the ATPMS-IGZO film is then immersed in, for example, 20 mM glutaraldehyde (GA, OHC(CH₂)₃CHO) in PBS solution for some time (e.g., 2 hours). In some embodiments, GA acts as the cross-linker molecule to immobilize the sensing enzyme glucose oxidase. In some embodiments, IGZO-FET device is transferred and kept in 10 g/L glucose oxidase (GOx) in PBS for some time (e.g., 2 hours). The sample is rinsed with water and then dried with flowing nitrogen prior to electrical measurements, in accordance with some embodiments.

In some embodiments, Glucose Detection is performed as follows. All IGZO-FET electrical measurements are performed in the dark at room temperature using an Agilent 4155C precision semiconductor parameter analyzer. Forward (e.g., low voltage to high voltage) and backward (e.g., high voltage to low voltage) sweep drain-to-source current versus gate voltage (I_{D}-V_{G}), transfer curves are measured with the drain voltage (V_{D}) set to 100 mV at a V_{G} sweep step of 0.2 V and a sweep rate of V_{G}=0.2 V·s⁻¹. A PDMS well is attached to the top of the exposed IGZO channel, in accordance with some embodiments. Aqueous solution with/without glucose is purged with Ar in order to reduce oxygen levels to that of mammalian interstitial fluid (e.g., 45 ton or 0.08 mM). Solutions are introduced into the PDMS well using a syringe, where the glucose concentration is varied between 0-32 mM, which corresponds to the relevant clinical interstitial (2-30 mM) and tear (0.35±0.04 mM) fluid glucose levels of diabetic patients.

The following section illustrates results of various embodiments. Referring back to FIG. 5 transfer characteristics of IGZO-FETs under different surface functionalizations are compared. These measurements are performed in air, where I_{D} is measured while V_{G} is scanned from −10 V up to 25 V and back down to −10 V with a constant V_{D} set to 100 mV. The IGZO-FETs have good electrical characteristics and may not have significant hysteresis between the up and down sweeps of V_{G} in the I_{D}-V_{G} data suggesting traps are able to stay in equilibrium with the sweep rate of V_{G}.

A summary of the data is given in Table 2, where the average electron mobility (μ_{avg}), turn-on voltage (V_{ON}), drain current on-to-off ratio (I_{ON}/I_{OFF}), and hysteresis for devices with different surface treatments (e.g., IGZO FETs without passivation, with APTMS and GOx. (V_{D}=100 mV)) are determined.

TABLE 2

| Surface | | | | |
|---|---|---|---|---|
| treatment | μ_{avg} (cm²/Vs) | V_{ON} (V) | I_{ON}/I_{OFF} | Hysteresis (V) |
| IGZO-FET | 14.6 | 0.5 | 2.8 × 10⁵ | 0.6 |
| APTMS | 14.5 | 0.5 | 1.5 × 10⁵ | 3.3 |
| GOx | 14.1 | 0.5 | 1.4 × 10⁵ | 5.4 |

Both μ_{avg} and on-voltage V_{ON} are extracted using methods described previously. The IGZO-FETs have high ratio of on current to off current (I_{ON}/I_{OFF} ratio) approximately 105, due in part to low I_{OFF}, which is very important for sensors, a high μ_{avg} greater than 14 cm²/Vs, and low gate leakage currents (I_{G} approximately 10-11 A). The major difference after adsorption of APTMS or GOx on the back channel surface is a slight decrease in μ_{avg}, and I_{ON}/I_{OFF}, which can be correlated with an increase in hysteresis.

FIGS. 6B, 7B, and 8B illustrate cross-sections of various embodiments 620, 720, and 820 of top-gated IGZO-FET structures, respectively, according to some embodiments. Materials for various layers/structures in the top-gated IGZO-FET structures are similar to the ones described wherein reference to bottom-gated IGZO-FET structures of FIGS. 6A, 7A, and 8A. In various embodiments of FIGS. 6B, 7B, and 8B the enzyme or chemical is adjacent to the gate (e.g., on top of the gate).

FIG. 9A illustrates a 3D illustration of an apparatus 900 comprising an IGZO-FET sensor, in accordance with some embodiments. FIG. 9B illustrates an optical image 920 of IGZO-FET device with W/L ratio of 100 μm/20 μm, in accordance with some embodiments. Apparatus 900 shows an IGZO-FET with inlet 901 and outlet 902 to pass through the material to-be sensed (e.g., glucose), IGZO sensing material 903 to detect material 904, substrate 601, gate 602, dielectric (e.g., SiO₂) 603, source/drain 604a/b, and PDMS 101.

In some embodiments, a PDMS well 905 is used to define the contact area of the analyze solution and the back channel of the IGZO-FET. First, the response of APTMS functionalized IGZO-FET to pH is detected, in accordance with some embodiments. In some embodiments, when the applied gate voltage V_{G} exceeds V_{ON}, electrons are injected from the source electrode 604a to the drain electrode 604b through electron accumulation region of IGZO thin-film 903 induced by the positive gate bias at the interface between IGZO active layer 903 and SiO₂ gate insulator 603.

The adsorption of protons to the aminosilane groups enable protonation of —NH₂ to —NH₃⁺ on the silanized IGZO surface. The positively charged aminosilane groups onto IGZO surface introduce acceptor-like surface state which can capture electrons from IGZO conduction band and deplete electron carriers in n-type IGZO film below, thereby increasing the surface band bending. This leads to conductance decrease and a more positive V_{G} is needed to bend the conduction band at SiO₂/IGZO interface as shown in FIGS. 10A-B. FIGS. 10A-B illustrate plots 1000 and 1020, respectively, showing IGZO-FET backchannel functionalized with aminopropyltrimethoxysilane (ATPMS) exposed to varying pH, and source (S) and drain (D) charge current vs. pH, in accordance with some embodiments.

Referring back to FIGS. 9A-B, the conductance between S/D contacts 604a/b is increased in proportional to pH with a sensitivity of approximately 1.0×10⁻⁷ A/pH. Then, a phosphate buffered saline (PBS) solution (composed of, for example, 137 mM NaCl, 2.5 mM KCl, 4 mM NaH₂PO₄, and 16 mM Na₂HPO₄, pH 7.4) is used to dilute glucose to the range of concentrations of interest.

FIG. 11A illustrates plot 1100 of an IGZO-FET backchannel functionalized with GOx exposed to varying concentrations of glucose, inset, and including a schematic diagram showing the role of positively charged aminosilane groups as an electron acceptor, according to some embodiments. Transfer characteristics 1100 are shown in FIG. 11A for IGZO-FETs functionalized with GOx enzyme, and with varying concentration of glucose, in accordance with some embodiments.

FIG. 11B illustrates a schematic diagram 1120 showing the impact of positively charged aminosilane groups on band bending at IGZO surface, according to some embodiments.

In some embodiments, the I_{ON}/I_{OFF} ratio of the devices in solution is decreased to approximately 10³, primarily due to an increase in I_{OFF}. In some embodiments, V_{ON} is shifted to positive values with increasing glucose concentration. One reason for such shift can be attributed to the reaction between GOx enzyme and glucose. Glucose is biocatalytically oxidized and forms gluconic acid in the presence of GOx, which can result in the acidification at IGZO/electrolyte interface through proton dissociation. In some embodiments, the generated protons (e.g., lower pH) in the vicinity of pH-sensitive aminosilane groups induce a more positive V_{ON} and decrease S/D conductance of the underlying IGZO-FETs as mentioned herein. To quantify the sensor response to glucose, I_{D} is measured with V_{G} set to 11 V, which is the maximum in sub-threshold slope for the IGZO-FET in PBS solution.

FIG. 5A illustrates plot 1200 showing time course of S/D current change for varying concentrations of glucose (C_{glucose}) at a fixed V_{G} as measured using IGZO FET functionalized with GOx, where arrows 1 and 2 indicate addition of 0.13 mM acetaminophen and ascorbic acid, respectively, according to some embodiments. Significant changes in I_{D} are observed in this glucose concentration range and the current decreases/increases in a stepwise fashion as C_{glucose} is increased or decreased. In some embodiments, the conductance changes are fully reversible for increasing and/or decreasing C_{glucose}. The response time for glucose sensing is measured as less than 10 s. The arrows in FIG. 12A indicate when interfering compounds (acetaminophen and ascorbic acid) are added to the electrolyte at the end of the experiment. In some embodiments, interference of acetaminophen/ascorbic acid is totally suppressed. This suggests that GOx functionalized IGZO-FET selectively detects glucose only and that the device is effective in minimizing interference from acetaminophen/ascorbic acid, in accordance with some embodiments. This can be attributed to the specific catalysis reaction between GOx enzyme and glucose.

FIG. 12B illustrates plot 1220 showing S/D current change vs. logarithmic glucose concentrations, according to some embodiments of the disclosure. Here, the change in S/D current is plotted versus C_{glucose}, and a linear relationship is obtained for the semi-log plot, in accordance with some embodiments. The slope of AID change versus log(C_{glucose}) is −2.2×10⁻⁸ A·mM⁻¹ and a coefficient of determination (R²) of 0.999 is found for C_{glucose} up to 28 mM. These data suggest that several complimentary measurements allow the determination of glucose concentrations, and the combination of measuring I_{D} at a fixed V_{G} may allow improved accuracy for sensing of glucose.

In some embodiments, a transparent FET sensor with nano-structured amorphous In—Ga—Zn—O Wires is described. Following are source of materials associated with transparent FET sensor with nano-structured amorphous In—Ga—Zn—O Wires, in accordance with some embodiments.

Glucose is obtained from Alfa Aesar; HCl, NaCl, KCl, NaH₂PO₄, Na₂HPO₄ are acquired from Macron; Aminopropyltrimethoxysilane, polystyrene nanospheres, acetaminophen and ascorbic acid are from Sigma-Aldrich; Glutaraldehyde is acquired from Electron Microscopy Sciences; Glucose oxidase is obtained from Amresco. IGZO and ITO targets are from AJA International Inc. and Kurt J. Lesker Inc., respectively; Photoresist S1818 and SU-8 are acquired from Microchem; Sylgard 184 PDMS is obtained from Dow Corning; and Milli-Q water (18.2 MΩ cm) is used in all sample preparation.

The following section describes the fabrication of a transparent FET sensor with nano-structured amorphous In—Ga—Zn—O wires, in accordance with some embodiments. In some embodiments, an IGZO-FET test structure is fabricated using a heavily p-doped Si substrate as the gate and thermally grown Sift (e.g., 100 nm thick) as the gate dielectric. In some embodiments, ITO films (e.g., 160 nm thick, measured by ellipsometry) are deposited on Si/SiO₂ substrate using RF magnetron sputter deposition with a 3 inch ITO target (e.g., composition: In₂O₃:SnO₂=90:10 wt %), 120 W RF power, approximately 4 mTorr chamber pressure, and 20 sccm flow rate of Ar.

In some embodiments, source and drain electrodes 604a/b are patterned using photolithography and etched in HCl (1:20 in DI) giving a channel length of, for example, 20 μm. In some embodiments, the ITO films are then annealed at 300° C. for one hour to increase their resistance to HCl etch, increase their electrical conductivity, and improve their transparency. In some embodiments, Polystyrene (PS) nanospheres (500 nm) in mixture of EtOH/water (1:2) is deposited by spin coating onto channel defined by ITO S/D electrodes to form a hexagonal close-packed monolayer on the substrates. In some embodiments, the spin-coating process comprises the following three stages: (a) 300 rpm for 10 sec to spread the bead solution evenly (b) 450 rpm for 3 min to spin away the excess bead solution and (c) 1400 rpm for 10 sec to spin off the excess materials from the edges. In some embodiments, oxygen plasma is employed to shrink the size of PS nanospheres.

In some embodiments, amorphous IGZO films 903 (e.g., approximately 50 nm thick) are deposited by sputter deposition with a 3 inch IGZO sputter target (e.g., molar composition: In₂O₃:Ga₂O₃:ZnO), 100 W RF power, approximately 4 mTorr chamber pressure, and 20 sccm flow rate with a 1:19 (O₂:Ar) ratio.

In some embodiments, PS nanospheres are removed by ultrasonication in CH₂Cl₂ for 2 minutes leading to closely packed hexagonal IGZO nanostructures. In some embodiments, the fabricated nanostructured IGZO channel is patterned to be a wire by electrohydrodynamic printing of SU-8 photoresist followed by etching. In some embodiments, the etching solution is diluted HCl in DI (1:200). In some embodiments, the fabricated IGZO-FETs are subsequently annealed in air at 300° C. to improve the device performance.

The following section describes the surface functionalization of the transparent FET sensor with nano-structured amorphous In—Ga—Zn—O wires, in accordance with some embodiments. In some embodiments, IGZO surface is cleaned in oxygen plasma (e.g., 50 W power for 2 minutes) to remove contaminants. In some embodiments, the IGZO surface is immediately soaked in 1% ethanol solution of aminopropyltrimethoxysilane (APTMS) for 2 hours, rinsed with ethanol, and then dried with flowing nitrogen. In some embodiments, the ATPMS-IGZO film is then immersed in 20 mM glutaraldehyde (GA) in PBS solution for 2 hours. GA acts as the cross-linker molecule to immobilize the sensing enzyme glucose oxidase. In some embodiments, the device is transferred and kept in 10 g/L glucose oxidase (GOx) in PBS for 2 hours. In some embodiments, the sample is rinsed with water and then dried with flowing nitrogen prior to electrical measurements.

The following section illustrates the glucose detection process of the transparent FET sensor with nano-structured amorphous In—Ga—Zn—O wires, in accordance with some embodiments.

In some embodiments, IGZO-FET electrical measurements are performed in the dark at room temperature using an Agilent 4155C precision semiconductor parameter analyzer. Forward (low voltage to high voltage) and backward (high voltage to low voltage) sweep drain-to-source current versus gate voltage (I_{D}-V_{G}) transfer curves are measured with the drain voltage (V_{D}) set to 100 mV at a V_{G} sweep step of 0.2 V and a sweep rate of V_{G}=0.2 V·s⁻¹. In some embodiments, a PDMS well 905 is attached to the top of the exposed IGZO channel 903. In some embodiments, an aqueous solution with/without glucose is purged with Ar to reduce oxygen levels to that of mammalian interstitial fluid (45 torr or 0.08 mM). In some embodiments, solutions are introduced into the PDMS well using a syringe, where the glucose concentration is varied between 0-32 mM, which corresponds to the relevant clinical interstitial (2-30 mM) and tear (0.35±0.04 mM) fluid glucose levels of diabetic patients.

The following section illustrates the process of and results of glucose detection by the transparent FET sensor with nano-structured amorphous In—Ga—Zn—O wires, in accordance with some embodiments. In some embodiments, the method of forming nanostructures is colloidal nano-sphere lithography. Other methods of nanopatterning may including self-assembly, e-beam lithography, nano-imprint lithography, interference lithography and scanning probe lithography.

FIG. 13A illustrates optical images 1300 (images 1301, 1302, and 1303) of polystyrene sphere monolayer on Sift/Si substrate after oxygen plasma treatment, according to some embodiments of the disclosure. In some embodiments of colloidal nano-sphere lithography, as the solvent evaporates, capillary forces draw the PS nanospheres together, and the nanospheres pack in a hexagonally close-packed pattern, tightly attaching on the substrates as shown in FIG. 13A. In some embodiments, oxygen plasma is used to shrink the size of PS nanospheres prior to IGZO deposition.

FIG. 13B illustrates pictures 1320 (e.g., images 1321, 1322, 1323) of IGZO nanostructure on Sift/Si substrate after PS liftoff, according to some embodiments of the disclosure. In some embodiments, the preferred pattern of IGZO nanowires are obtained from 70 seconds of oxygen plasma treatment as shown FIG. 13B. In one example, discontinuous wire or continuous film with random defects are obtained for 50 or 90 seconds treatment, respectively.

In some embodiments, the same procedure is used to deposit and pattern IGZO nanostructures as an active channel as shown in FIG. 14A and FIG. 14B.

FIGS. 14A-D illustrate optical images 1400, 1420, 1430, and 1440 of PS monolayer on ITO S/D by spin coating, nanostructured IGZO film on ITO S/D after PS liftoff, E-jet printed SU-8 wire on nanostructured IGZO film, and final device after IGZO etching, SU-8 developing and oxygen plasma cleaning, respectively, in accordance with some embodiments. In some embodiments, the channel is further patterned into 8 μm wide wire by E-jet printing and acid etching are shown in FIG. 14C and FIG. 14D. In this example, the hexagonal IGZO nanowires can be clearly identified between ITO S/D electrodes by SEM and AFM.

FIGS. 14E-F illustrate an SEM image 1450 of nanostructured IGZO wire as channel between ITO S/D electrodes, and AFM image 1460 of IGZO closely packed hexagonal nanowires (3×3 μm²), according to some embodiments of the disclosure.

FIG. 15 illustrates a plot 1500 showing transfer characteristics of nanostructured IGZO-FET, in accordance with some embodiments. In some embodiments, the electric performance of the fabricated FET device is measured by sweeping I_{D} vs V_{G} with a constant V_{D} as shown in FIG. 15. In some embodiments, the FETs may not have significant hysteresis between the up and down sweeps, suggesting traps have the ability to stay in equilibrium with the sweep rate of V_{G}=0.2 V/s. A reasonable I_{ON}/I_{OFF} ratio of approximately 10³ and μ_{avg} approximately 3.6 cm²/Vs are obtained, while the gate leakage currents (I_{G}) are typically less than 1 nA. This indicates that patterned materials of various embodiments are robust for device integration, have good semiconductor/source and drain interfaces, and have high-quality dielectric/channel interface.

FIG. 16A illustrates a plot 1600 showing IGZO-FET backchannel functionalized with GOx exposed to varying concentrations of glucose, according to some embodiments. FIG. 16B illustrates a schematic diagram 1620 showing the role of positively charged aminosilane groups as an electron acceptor and its impact on band bending at IGZO surface, in accordance with some embodiments. FIG. 16A illustrates the response of the nanostructured IGZO-FETs functionalized with GOx enzyme to varying concentration of glucose, in accordance with some embodiments. In some embodiments, V_{ON} shifted to positive values and a decreased S/D conductance with increasing glucose concentration is observed. This observation can be attributed to the specific interaction between GOx enzyme and glucose, in accordance with some embodiments. Glucose is biocatalytically oxidized and forms gluconic acid in the presence of GOx, which can result in the acidification at IGZO/electrolyte interface through proton dissociation. The generated protons (lower pH) in the vicinity of pH-sensitive aminosilane groups enable protonation of —NH₂ to —NH₃⁺. The positively charged aminosilane groups deplete electron carriers in n-type IGZO film, thereby inducing a more positive V_{ON} and decreasing S/D conductance of the underlying nanostructured IGZO-FETs, in accordance with some embodiments.

FIG. 6A illustrates a plot 1700 showing transfer characteristics of an IGZO FET backchannel functionalized with GOx exposed to varying concentrations of glucose.

FIG. 17B illustrates a plot 1720 showing difference in drain current (AID) versus time for various glucose concentrations as measured using a nanostructured IGZO-FET functionalized with GOx, in accordance with some embodiments.

FIG. 17D illustrates a plot 1740 showing difference in drain current (AID) versus time for various glucose concentrations as measured using a solid IGZO-FET functionalized with GOx, where arrows 1 and 2 indicate the addition of 0.13 mM acetaminophen and ascorbic acid, respectively, in accordance with some embodiments.

Plots 1720 and 1740 illustrate continuous glucose monitoring which is conducted by measuring I_{D} with constant V_{G} and V_{D}, in accordance with some embodiments. In some embodiments, I_{D} decreases/increases in a stepwise fashion in this glucose concentration range as glucose concentration (C_{glucose}) is increased or decreased. In some embodiments, the conductance changes are fully reversible for increasing and/or decreasing C_{glucose}. The response time for glucose sensing is less than 10 s, in accordance with some embodiments. In some embodiments, the high surface/volume ratio of IGZO wires enables the detection limit for nanostructured IGZO FET to be as low as 10 μM (micro molar) in tears, which is suitable for glucose sensing in tears. In some embodiments, the change in S/D current has a linear relationship versus C_{glucose} as shown in FIG. 17C on a logarithmic scale. In some embodiments, interference effects from acetaminophen/ascorbic acid are totally suppressed by the highly selective glucose sensor.

FIG. 17C illustrates a plot 1730 showing difference in drain current (AID) versus time for various glucose concentrations as measured using |ΔIds| versus C_{glucose} on a logarithmic scale, in accordance with some embodiments.

Some embodiments describe an inverter which with at least one TFT (transparent FET) sensitized with GOx and the other passivated with a self-assembled monolayer, inactive GOx, or inorganic layer. As such, more sensing sensitivity can be achieved.

While some embodiments are described with reference to one sensor, an array of sensors can be formed that are functionalized for a range of analytes, in accordance with some embodiments. The sensors of various embodiments are transparent and use a field effect sensing method. Transparency allows multiple sensors to be integrated in the field of view and the field effect approach results in larger signals when the sensors are reduced in size. Both of these factors provide more real estate for the sensing capabilities, in accordance with some embodiments.

Further, although contact lenses are one application discussed, other applications of various embodiments include simultaneous electrophysiological recording and neural imaging, integrated sensing on an endoscope, and pressure sensors for medical applications and soft robotics. While for contact lens, some embodiments describe glucose sensing. However, the embodiments are not limited to such. For instance, a range of other biomaterial sensing is possible by monitoring a patients' tears. Examples include other diseases (e.g., liver disease, glaucoma, cardiovascular, renal, etc.), potentially for cancer diagnostics with an adequate array of sensors, monitoring drug metabolites to confirm patients are taking their required medication treatments (e.g., malaria vaccines, medication for post-traumatic stress, etc.), stress markers (e.g., testosterone, cortisol, etc.), and many other applications.

FIG. 18 illustrates a sensing array of pixels 1800, according to some embodiments of the disclosure. In this example, a 10×10 pixel array is shown with 10 gate, source, and drain wires connected to the respective 10 gate, source, and drain terminals of the transparent active devices.

FIG. 19 illustrates a top view of an IGZO-FET pixel in a sensing array 1900 showing the source 604a, drain 604b, and gate 602 terminals, and location of the sensing enzyme on the IGZO 903, in accordance with some embodiments. The layout of IGZO-FET 1900 shows that the location of the sensing material is on IGZO 903. In some embodiments, electrical connections to 604a, 604b, and 602 can be made at the edge of the array and each pixel in the sensing array is addressed using the controller 405.

FIG. 20 illustrates a single sensing pixel 2000 of the array of FIG. 18, in accordance with some embodiments. Compared to FIG. 19, here the sensing enzyme is on the floating gate 602. In some embodiments, electrical connections to 604a and 604b (source/drain) can be made at the edge of the array and each pixel in the sensing array is addressed using the controller 405.

Reference in the specification to “an embodiment,” “one embodiment,” “some embodiments,” or “other embodiments” means that a particular feature, structure, or characteristic described in connection with the embodiments is included in at least some embodiments, but not necessarily all embodiments. The various appearances of “an embodiment,” “one embodiment,” or “some embodiments” are not necessarily all referring to the same embodiments. If the specification states a component, feature, structure, or characteristic “may,” “might,” or “could” be included, that particular component, feature, structure, or characteristic is not required to be included. If the specification or claim refers to “a” or “an” element, that does not mean there is only one of the elements. If the specification or claims refer to “an additional” element, that does not preclude there being more than one of the additional element.

Furthermore, the particular features, structures, functions, or characteristics may be combined in any suitable manner in one or more embodiments. For example, a first embodiment may be combined with a second embodiment anywhere the particular features, structures, functions, or characteristics associated with the two embodiments are not mutually exclusive.

The following examples are provided with reference to various embodiments.

### Example 1

An apparatus comprising: a substrate comprising a transparent material; and an array of transparent active devices disposed on the substrate, wherein each transparent active device comprises a sensing element associated with the transparent active device.

### Example 2

The apparatus of example 1, where the sensing element comprises a component that recognizes an analyte and is attached to a semiconductor surface of the transparent active device.

### Example 3

The apparatus of example 1, where the sensing element comprises a component that recognizes an analyte and is attached to a transparent active device gate electrode of the transparent active device.

### Example 4

The apparatus according to any one of examples 2 or 3, wherein the component comprises one of: enzymes or chemicals to detect one or more of glucose; acid including deoxyribonucleic acid (DNA) or ribonucleic acid (RNA); proteins; lipids; or carbohydrates.

### Example 5

The apparatus according to any one of examples 1 to 3, wherein the substrate is conformed to a non-flat shape.

### Example 6

The apparatus of example 1 comprises a first lens disposed over the array of transparent active devices; and a second lens disposed under the substrate.

### Example 7

The apparatus of example 1 comprises an array of antennas disposed on the substrate.

### Example 8

The apparatus of example 7, wherein the array of antennas comprises graphene.

### Example 9

The apparatus of example 7, wherein the array of antennas is invisible.

### Example 10

The apparatus of example 7 comprises a controller coupled to the array of transparent active devices.

### Example 11

The apparatus of example 10 comprises a power source coupled to the controller.

### Example 12

The apparatus of example 11, wherein the power source comprises a capacitor which is to be charged by wireless means.

### Example 13

An apparatus comprising: a substrate; a gate above the substrate; a dielectric above the gate; an active region over the dielectric; a source adjacent to the active region; a drain adjacent to the active region, wherein the source and drain are on either side of the active region; and a sensing structure formed over the active region.

### Example 14

The apparatus of example 13, wherein at least one of the gate, source, and drain is formed of at least of one: transparent conducting oxides, conducting nanomaterials, organic conductors, polymers, or structured metal arrays.

### Example 15

The apparatus of example 14, wherein the transparent conducting oxides includes at least one or more of: In, Sn, 0, or Zn.

### Example 16

The apparatus of example 14, wherein the transparent conducting oxides includes at least one of: indium tin oxide (ITO), doped ZnO, doped SnO₂, zinc indium oxide (ZIO), or zinc tin oxide (ZTO).

### Example 17

The apparatus of example 14, wherein the conducting nanomaterials include at least one of: metal nanowires, carbon nanotubes, or graphene.

### Example 18

The apparatus of example 17, wherein the metal nanowires include one of: Ag, Au, or Cu.

### Example 19

The apparatus of example 14, wherein the organic conductors include one of: poly(3,4-ethylenedioxythiophene) (PEDOT), PDOT, or poly styrene sulfonate (PSS).

### Example 20

The apparatus of example 14, wherein the substrate comprises at least one of: polyethylene terephthalate (PET), polyethylene naphthalate (PEN), polycarbonate (PC), polyimide (PI), cellulose based substrates, collagen based substrates, or glass.

### Example 21

The apparatus of claim 13, wherein the active region comprises at least one of: In, Ga, Zn, O, or Sn.

### Example 22

The apparatus of claim 13, wherein the active region comprises: In_{w}GaₓZn_{y}O_{z}; SnₓZn_{y}O_{z}; InₓZn_{y}O_{z}; InₓGa_{y}O_{z}; or In_{w}SnₓZn_{y}O_{z}.

### Example 23

The apparatus of claim 13, wherein the active region comprises of at least one of: ZnO, In₂O₃, SnO₃, Ga₂O₃, or combinations thereof.

### Example 24

The apparatus according to any one of examples 13 to 23, wherein the sensing structure comprises at least one of: glucose oxidase, urease, invertase, mutarotase, maltase, alcohol dehydrogenase, aldehyde dehydrogenase, cortisol specific monoclonal antibody, anti-testosterone monoclonal antibody, or peroxidase.

### Example 25

The apparatus of example 13, wherein the active region comprises nanostructures.

### Example 26

The apparatus of example 25, wherein the nanostructures comprises Indium-Gallium-Zinc-Oxide (IGZO) nanostructures.

### Example 27

The apparatus of example 25, wherein the nanostructures are one of: hexagonal nanowires, parallel lines, square grids, diamond grids, or spiral structures.

### Example 28

A method comprising: forming a substrate comprising a transparent material; and disposing an array of transparent active devices on the substrate, wherein each transparent active device comprises a sensing element associated with the transparent active device.

### Example 29

The method of example 28 where the sensing element comprises an enzyme attached to a semiconductor surface of the transparent active device.

### Example 30

The method of example 28 where the sensing element comprises an enzyme attached to a transparent active device gate electrode of the transparent active device.

### Example 31

The method of example 28, wherein the substrate is conformed to a non-flat shape.

### Example 32

The method of example 28 comprises disposing a first lens over the array of transparent active devices.

### Example 33

The method of example 32 comprises disposing a second lens under the substrate.

### Example 34

The method of claim 28 comprises disposing an array of antennas on the substrate.

### Example 35

The method of claim 34 wherein the array of antennas comprise graphene.

### Example 36

The method of claim 34, wherein the array of antennas is invisible.

### Example 37

The method according to any one of examples 28 to 34 comprises coupling a controller to the array of transparent active devices.

### Example 38

The method of example 37 comprises coupling a power source coupled to the controller.

### Example 39

The method of example 38 comprises charging, by wireless means, a capacitor of the power source.

### Example 40

A method comprising: forming a substrate; forming a gate above the substrate; forming a dielectric above the gate; forming an active region over the dielectric; forming a source adjacent to the active region; forming a drain adjacent to the active region, wherein the source and drain are on either side of the active region; and forming a sensing structure formed over the active region.

### Example 41

The method of example 40, wherein forming one of the gate, source, and drain comprises forming at least of one: transparent conducting oxides, conducting nanomaterials, organic conductors, polymers, or structured metal arrays.

### Example 42

The method of example 41, wherein the transparent conducting oxides include at least one or more of: In, Sn, O, or Zn.

### Example 43

The method of example 41, wherein the transparent conducting oxides includes at least one of: indium tin oxide (ITO), doped ZnO, doped SnO₂, zinc indium oxide (ZIO), or zinc tin oxide (ZTO).

### Example 44

The method of example 41, wherein the conducting nanomaterials include at least one of: metal nanowires, carbon nanotubes, or graphene.

### Example 45

The method of example 41, wherein the metal nanowires include one of: Ag, Au, or Cu.

### Example 46

The method of example 41, wherein the organic conductors include one of: poly(3,4-ethylenedioxythiophene) (PEDOT), PDOT, or poly styrene sulfonate (PSS).

### Example 47

The method of example 41, wherein the substrate comprises at least one of: polyethylene terephthalate (PET), polyethylene naphthalate (PEN), polycarbonate (PC), polyimide (PI), cellulose based substrates, collagen based substrates, or glass.

### Example 48

The method of example 40, wherein the active region comprises at least one of: In, Ga, Zn, O, or Sn.

### Example 49

The method of example 40, wherein the active region comprises: In_{w}GaₓZn_{y}O_{z}; SnₓZn_{y}O_{z}; InₓZn_{y}O_{z}; InₓGa_{y}O_{z}; or In_{w}SnₓZn_{y}O_{z}.

### Example 50

The method of example 40, wherein the active region comprises of at least one of: ZnO, In₂O₃, SnO₂, Ga₂O₃, or combinations thereof.

### Example 51

The method of example 40, wherein the sensing structure comprises at least one of: glucose oxidase, urease, invertase, mutarotase, maltase, alcohol dehydrogenase, aldehyde dehydrogenase, cortisol specific monoclonal antibody, anti-testosterone monoclonal antibody, or peroxidase.

### Example 52

The method of example 40, wherein the active region comprises nanostructures.

### Example 53

The method of example 52, wherein the nanostructures comprises Indium-Gallium-Zinc-Oxide (IGZO) nanostructures.

### Example 54

The method of example 52, wherein the nanostructures are one of: hexagonal nanowires, parallel lines, square grids, diamond grids, or spiral structures.

While the disclosure has been described in conjunction with specific embodiments thereof, many alternatives, modifications and variations of such embodiments will be apparent to those of ordinary skill in the art in light of the foregoing description. The embodiments of the disclosure are intended to embrace all such alternatives, modifications, and variations as to fall within the broad scope of the appended claims.

In addition, well known power/ground connections to integrated circuit (IC) chips and other components may or may not be shown within the presented figures, for simplicity of illustration and discussion, and so as not to obscure the disclosure. Further, arrangements may be shown in block diagram form in order to avoid obscuring the disclosure, and also in view of the fact that specifics with respect to implementation of such block diagram arrangements are highly dependent upon the platform within which the present disclosure is to be implemented (i.e., such specifics should be well within purview of one skilled in the art). Where specific details (e.g., circuits) are set forth in order to describe example embodiments of the disclosure, it should be apparent to one skilled in the art that the disclosure can be practiced without, or with variation of, these specific details. The description is thus to be regarded as illustrative instead of limiting.

An abstract is provided that will allow the reader to ascertain the nature and gist of the technical disclosure. The abstract is submitted with the understanding that it will not be used to limit the scope or meaning of the claims. The following claims are hereby incorporated into the detailed description, with each claim standing on its own as a separate embodiment.

1-54. (canceled)

55. An apparatus comprising:
a substrate comprising a transparent material; and
an array of transparent active devices disposed on the substrate, wherein each transparent active device comprises a sensing element associated with the transparent active device.

56. The apparatus of claim 55 where the sensing element comprises a component that recognizes an analyte and is attached to a semiconductor surface of the transparent active device.

57. The apparatus of claim 55 where the sensing element comprises a component that recognizes an analyte and is attached to a transparent active device gate electrode of the transparent active device.

58. The apparatus of claim 56, wherein the component comprises one of: enzymes or chemicals to detect one or more of: glucose; acid including deoxyribonucleic acid (DNA) or ribonucleic acid (RNA); proteins; lipids; or carbohydrates.

59. The apparatus of claim 55, wherein the substrate is conformed to a non-flat shape.

60. The apparatus of claim 55 comprises:
a first lens disposed over the array of transparent active devices; and
a second lens disposed under the substrate.

61. The apparatus of claim 55 comprises an array of antennas is on the substrate.

62. The apparatus of claim 61, wherein the array of antennas comprises graphene.

63. The apparatus of claim 61, wherein the array of antennas is invisible.

64. The apparatus of claim 61 comprises a controller coupled to the array of transparent active devices.

65. The apparatus of claim 64 comprises a power source coupled to the controller.

66. The apparatus of claim 65, wherein the power source comprises a capacitor, which is to be charged by wireless means.

67. An apparatus comprising:
a substrate;
a gate above the substrate;
a dielectric above the gate;
an active region over the dielectric;
a source adjacent to the active region;
a drain adjacent to the active region, wherein the source and drain are on either side of the active region; and
a sensing structure formed over the active region.

68. The apparatus of claim 67, wherein at least one of the gate, source, and drain is includes least of one:
transparent conducting oxides,
conducting nanomaterials,
organic conductors, polymers, or
structured metal arrays.

69. The apparatus of claim 68, wherein the transparent conducting oxides includes at least one or more of: In, Sn, O, or Zn.

70. The apparatus of claim 68, wherein:
the transparent conducting oxides includes at least one of: indium tin oxide (ITO), doped ZnO, doped SnO₂, zinc indium oxide (ZIO), or zinc tin oxide (ZTO);
the conducting nanomaterials include at least one of: metal nanowires, carbon nanotubes, or graphene;
the metal nanowires include one of: Ag, Au, or Cu; and
the organic conductors include one of: poly(3,4-ethylenedioxythiophene) (PEDOT), PDOT, or poly styrene sulfonate (PSS).

71. The apparatus of claim 68, wherein:
the substrate comprises at least one of: polyethylene terephthalate (PET), polyethylene naphthalate (PEN), polycarbonate (PC), polyimide (PI), paralene, hydrogel, cellulose based substrates, collagen based substrates, or glass; and
the active region comprises at least one of: In, Ga, Zn, O, or Sn.

72. The apparatus of claim 67, wherein the sensing structure comprises at least one of: glucose oxidase, urease, invertase, mutarotase, maltase, alcohol dehydrogenase, aldehyde dehydrogenase, cortisol specific monoclonal antibody, anti-testosterone monoclonal antibody, or peroxidase.

73. The apparatus of claim 67, wherein the active region comprises nanostructures.

74. The apparatus of claim 73, wherein the nanostructures comprises Indium-Gallium-Zinc-Oxide (IGZO) nanostructures.

75. The apparatus of claim 73, wherein the nanostructures are one of: hexagonal nanowires, parallel lines, square grids, diamond grids, or spiral structures.

76. A method comprising:
forming a substrate;
forming a gate above the substrate;
forming a dielectric above the gate;
forming an active region over the dielectric;
forming a source adjacent to the active region;
forming a drain adjacent to the active region, wherein the source and drain are on either side of the active region; and
forming a sensing structure formed over the active region.

77. The method of claim 76, wherein forming one of the gate, source, and drain comprises forming at least of one:
transparent conducting oxides,
conducting nanomaterials,
organic conductors, polymers, or
structured metal arrays.
